(19) United States
(12) Patent Application Publication
(10) Pub. No.: US 2026/0272908 A1
(43) Pub. Date: Sep. 17, 2026

(54) PYRVINIUM AND ITS DERIVATIVES FOR THE TREATMENT OF PRE-CANCERS

(71) Applicants: VANDERBILT UNIVERSITY, Nashville, TN (US); The United States Government as represented by the Department of Veterans Affairs, Washington, DC (US)

(72) Inventors: Eunyoung Choi, Nashville, TN (US); James Goldenring, Nashville, TN (US)

(21) Appl. No.: 19/471,647

(22) PCT Filed: Apr. 5, 2024
(86) PCT No.: PCT/US2024/023198

§ 371 Date: Oct. 1, 2025

Related U.S. Application Data
(60) Provisional application No. 63/494,671, filed on Apr. 6, 2023.

(51) Int. Cl. A61K 31/4709; A61P 35/00

(52) U.S. Cl. CPC A61K 31/4709; A61P 35/00

(57) ABSTRACT

Described herein are pyrvinium and its derivatives for reprogramming a pre-cancerous mucosa to a non-cancerous state. Further described herein are pyrvinium and its derivatives for reducing the risk of cancer development in a living organism containing a metaplastic cell or a dysplastic cell.

### CROSS-REFERENCE TO RELATED APPLICATIONS

This application claims priority to U.S. Provisional Patent Application No. 63/494,671 filed on Apr. 6, 2023, which is incorporated by reference herein in its entirety.

### FEDERALLY SPONSORED RESEARCH

This invention was made with government support under grant numbers IBX000930 awarded by the Department of Veterans Affairs, CA190172 awarded by the Department of Defense, R01 DK101332, R01 CA272687, R37 CA244970 awarded by the National Institutes of Health. The government has certain rights in the invention.

### BACKGROUND

Gastric cancer is (GC) a significant global health concern, ranking as the fifth most common cancer and the third most common cause of cancer-related deaths. The risk of non-cardia GC is primarily associated with Helicobacter pylori (H. pylori) infection, which triggers a stepwise progression from atrophic gastritis, intestinal metaplasia (IM), to dysplasia. Population-based screening program have led to early detection and reduced mortality rates, and H. pylori eradication has been shown to reduce the risk of GC in nations with high incidence rates of GC, as well as the risk of metachronous GC in patients with early GC. However, long-term observational studies have demonstrated a high incidence of metachronous cancer even after H. pylori eradication. Furthermore, evidence suggests that patients with IM or dysplasia may not benefit from H. pylori treatment on the risk of GC. The concept of a “point of no return” suggests that GCs can occur after eradication of H. pylori if histologic changes are already advanced. Patients with GC often present with varying degrees of precancerous changes in gastric mucosa, such as IM and multifocal dysplastic glands. However, there are no therapeutic options available to treat asymptomatic patients with advanced precancerous lesions. Therefore, there is an urgent need to identify chemotherapeutic agents capable of effectively targeting these lesions.

What is needed are methods for using pyrvinium for the reprogramming of certain metaplastic and dysplastic cells to reverse their metaplastic/dysplastic phenotype, as well as inducing dysplastic cell death through either or both (a) blocking of MAPK signaling and/or (b) blocking of STAT3 signaling.

### SUMMARY

One embodiment described herein is a method for reprogramming a pre-cancerous mucosa to a non-cancerous state, the method comprising identifying a pre-cancerous mucosa comprising a metaplastic cell or a dysplastic cell; and contacting the metaplastic cell or the dysplastic cell with a compound of formula (I), or a salt thereof:

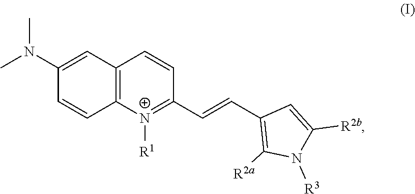

wherein:
R¹ is C₁₋₆ alkyl;
R^{2a} and R^{2b} are each independently C₁₋₆alkyl; and
R³ is C₆₋₁₂aryl, wherein R³ is optionally substituted with 1-5 R^{X}
R^{X}, at each occurrence, is independently C₁₋₆alkyl, C₁₋₄haloalkyl, halogen, cyano, —N(R^{Xa})₂,
   —OR^{Xb}, —SR^{Xb}, —C(O)R^{Xb}, —CO₂R^{Xb}, —C(O)N(R^{Xa})₂, —SO₂R^{Xa}, -L²-Y², —O-L^{X}-Y^{X}, —S-L^{X}-Y^{X}, or —N(R^{Xa})-L^{X}-Y^{X};
L^{X}, at each occurrence, is independently C₁₋₆alkylene, C₂₋₆alkenylene, or C₂₋₆alkynylene;
Y^{X}, at each occurrence, is independently hydrogen, cyano, halogen, haloalkyl, —OH, —N(R^{Xa})₂,
   —OR^{Xb}, —SR^{Xb}, —C(O)R^{Xb}, —CO₂R^{Xb}, —C(O)N(R^{Xa})₂, or —SO₂R^{Xa};
R^{Xa}, at each occurrence, is independently hydrogen, C₁₋₄alkyl, or —C(O)C₁₋₄alkyl; and
R^{Xb}, at each occurrence, is independently hydrogen, C₁₋₄alkyl, C₁₋₂haloalkyl, or —C(O)C₁₋₄alkyl.

In one aspect, the metaplastic cell or the dysplastic cell is contacted with a salt of the compound of formula (I).

In another aspect, the salt of the compound of formula (I) is a salt of formula (I-A),

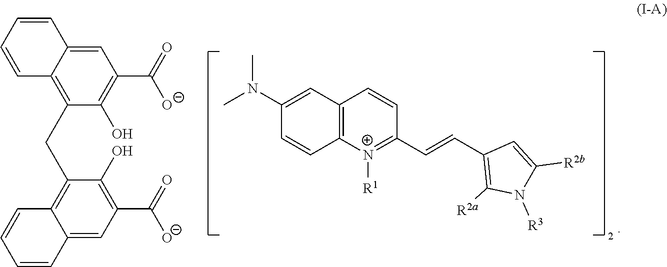

In another aspect, R¹ is methyl.

In another aspect, R^{2a} and R^{2b} are each methyl.

In another aspect, R³ is unsubstituted phenyl.

In another aspect, the compound of formula (I) is pyrvinium.

In another aspect, the metaplastic cell or the dysplastic cell is located within a living organism.

In another aspect, the pre-cancerous mucosa comprises a dysplastic cell.

In another aspect, the method comprises killing the dysplastic cell.

In another aspect, the dysplastic cell is positive for CD133 expression, CD166 expression, and/or Trop2 expression.

In another aspect, the dysplastic cell is a dysplastic gastric cell, a dysplastic esophageal cell, a dysplastic pancreatic cell, a dysplastic colon cell, or a dysplastic ovarian cell.

In another aspect, the dysplastic gastric cell is part of an organoid.

In another aspect, the pre-cancerous mucosa comprises a metaplastic cell.

In another aspect, the metaplastic gastric cell is positive for CD133, CD166, and/or Trop2 expression.

In another aspect, the metaplastic cell is a metaplastic gastric cell, a metaplastic esophageal cell, a metaplastic pancreatic cell, a metaplastic colon cell, or a metaplastic ovarian cell.

In another aspect, the metaplastic gastric cell is part of an organoid.

Another embodiment described herein is a method for reducing the risk of cancer development in a living organism containing a metaplastic cell or a dysplastic cell, the method comprising:
administering to the living organism a compound of formula (I), or a salt thereof:

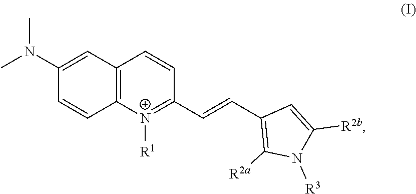

wherein:
R¹ is C₁₋₆alkyl;
R^{2a} and R^{2b} are each independently C₁₋₆ alkyl; and
R³ is C₆₋₁₂aryl, wherein R³ is optionally substituted with 1-5 R^{X}
R^{X}, at each occurrence, is independently C₁₋₆alkyl, C₁₋₄haloalkyl, halogen, cyano, —N(R^{Xa})₂,
   —OR^{Xb}, —SR^{Xb}, —C(O)R^{Xb}, —CO₂R^{Xb}, —C(O)N(R^{Xa})₂, —SO₂R^{Xa}, -L²-Y², —O-L^{X}-Y^{X},
   —S-L^{X}-Y^{X}, or —N(R^{Xa})-L^{X}-Y^{X};
L^{X}, at each occurrence, is independently C₁₋₆alkylene, C₂₋₆alkenylene, or C₂₋₆alkynylene;
Y^{X}, at each occurrence, is independently hydrogen, cyano, halogen, haloalkyl, —OH, —N(R^{Xa})₂,
   —OR^{Xb}, —SR^{Xb}, —C(O)R^{Xb}, —CO₂R^{Xb}, —C(O)N(R^{Xa})₂, or —SO₂R^{Xa};
R^{Xa}, at each occurrence, is independently hydrogen, C₁₋₄alkyl, or —C(O)C₁₋₄alkyl; and
   R^{Xb}, at each occurrence, is independently hydrogen, C₁₋₄alkyl, C₁₋₂haloalkyl, or —C(O)C₁₋₄alkyl.

In another aspect, a salt of the compound of formula (I) is administered to the living organism.

In another aspect, wherein the salt of the compound of formula (I) is a salt of formula (I-A),

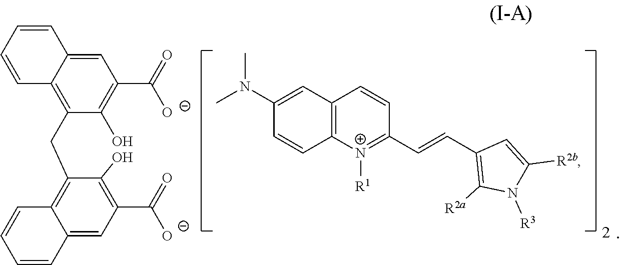

In another aspect, the compound of formula (I) is pyrvinium.

In another aspect, the method inhibits STAT3 and MEK/ERK signaling pathways in the metaplastic cell or the dysplastic cell.

In another aspect, the living organism is a non-human animal.

In another aspect, the living organism is a human.

In another aspect, the human is at risk for developing cancer.

In another aspect, the method further comprises administering a second agent to the living organism.

In another aspect, the second agent comprises a kinase inhibitor.

In another aspect, the kinase inhibitor comprises a MEK inhibitor.

In another aspect, the MEK inhibitor comprises trametinib, binimetinib, cobimetinib, and/or selumetinib.

Another embodiment described herein is the use of pyrvinium or a salt thereof in the manufacture of a medicament for reprograming a pre-cancerous mucosa to a non-cancerous state or reducing the risk of cancer development.

### DESCRIPTION OF THE DRAWINGS

FIG. 1A-1H show that pyrvinium induces cell death in mouse dysplastic organoids. FIG. 1A shows phase-contrast, Hematoxylin & Eosin (H&E) images, and co-immunostaining for CD44v9, Aqp5, and Trop2 in Meta3 and Meta4 organoids. FIG. 1B-1C show H&E and phase-contrast images of Meta3 (FIG. 1B) and 3 different Meta4 organoids (FIG. 1C) treated with DMSO vehicle, 1 μM of trametinib (Tra), 100 nM of pyrvinium (Pyr) and their combinations for 3 days. FIG. 1D-1E show quantitation of organoid diameters before and after treatment. (FIG. 1F) Phase-contrast images and live/dead (Calcein AM/EthD-1) cell staining with the percentages of live and dead cells in Meta4 organoid after treatment. FIG. 1G-1H show phase-contrast images (FIG. 1G) and quantitation of diameters (FIG. 1H) of Meta4 organoids before and after treatment with 0, 0.2, 0.5, and 1 μM of SSTC3, a specific Casein Kinase 1a activator. Mean±standard deviation. One-way ANOVA with Tukey's multiple comparisons. **P<0.01, ****P<0.0001. Scale bar=500 μM.

FIG. 2A-2D show that a phospho-antibody array identifies downregulation of MAPK and STAT signaling pathways by pyrvinium. FIG. 2A is a heat map showing pathway enrichment analysis of differentially phosphorylated proteins after pyrvinium (Pyr) treatment for 3 days in Meta3 and Meta4 organoids using the Molecular Signatures Database (MSigDB) hallmark gene set collection. Shading scale represents standardized log 2(FDR) values. FIG. 2B shows log 2 fold change bar graphs of signaling pathways in Meta3 and Meta4 organoids after treatment. FIG. 2C is a heat map showing the upregulated or downregulated phospho-proteins in Meta4 organoids compared to Meta3 after treatment. FIG. 2D graphically shows changes in phosphorylation of STAT3Y705 and ERKT202/T204 between Meta3 and Meta4 organoids (upper panel) or between DMSO and Pyr treatment in Meta4 organoid (lower panel). Each square represents the signal ratio of the antibody against the unphosphorylated form of the site. FIG. 2E schematically illustrates how the STAT3 and DPPs in RAS/MAPK pathway is affected by Pyr treatment in precancerous organoids.

FIG. 3A-3M show that pyrvinium suppresses mouse dysplastic organoids through double blockade of ERK and STAT3 signaling pathways. FIG. 3A-3B show the Western blot analysis for phospho-STAT3 (p-STAT3) and phospho-ERK (p-ERK) in three Meta4 organoids after treatment of trametinib (Tra), pyrvinium (Pyr) and their combinations for 3 days (FIG. 3A) and ratios of p-STAT3/total STAT3 and p-ERK/total ERK (FIG. 3B). FIG. 3C-3D show the Western blot analysis for p-STAT3 and p-ERK in Meta3 and Meta4 organoids (FIG. 3C) and ratios of p-STAT3/total STAT3 and p-ERK/total ERK (FIG. 3D). FIG. 3E shows immunostaining for p-STAT3 in Meta3 and Meta4 organoids. FIG. 3F-3G show phase-contrast images of Meta4 organoids treated with DMSO, Pyr (100 nM), STAT3-IN-1 (1 μM), Stattic (2 μM), and Cryptotanshinone (CPT) (5 μM) for 3 days (FIG. 3F), and quantitation of organoid diameters before and after treatment (FIG. 3G). FIG. 3H-3I show phase-contrast images of Meta4 organoids treated with DMSO, trametinib (Tra), STAT3-IN-1, combination of Tra and STAT3-IN-1, and Pyr, for 3 days (FIG. 3H), and quantitation of organoid diameters before and after treatment (FIG. 3I). FIG. 3J-3K show western blot for p-STAT3 and p-ERK after treatment of Tra, Pyr, and SSTC3 (FIG. 3J) and ratios of p-STAT3/t-STAT3 and p-ERK/t-ERK (FIG. 3K). FIG. 3L shows a heatmap of quantitative real time-PCR analysis for STAT3 target genes in Meta4 organoid treated with DMSO or Pyr for 1 day. Relative quantification of each gene expression level was normalized to GAPDH gene expression. Shading scale represents the mRNA expression values. FIG. 3M show representative mRNA of Pim-1 and Cyclin-B1 after treatment. Mean±standard deviation. Unpaired t-test or one-way ANOVA with Tukey's multiple comparisons. *P<0.05, **P<0.01, ***P<0.001, ****P<0.0001. ns, not significant. Scale bar=500 μM.

FIG. 4A-4J show that pyrvinium targets stem cell population in mouse dysplastic organoids. FIG. 4A-4B show a superimposed Uniform Manifold Approximation and Projection (UMAP) plot showing annotated clusters from DMSO-treated and pyrvinium (Pyr)-treated Meta4 cells (FIG. 4A) and subpopulation-matched cluster overlay (FIG. 4B). FIG. 4C shows PANTHER gene ontology classification results based on top 300 differentially expressed genes in DMSO-treated and Pyr-treated Meta4 cells. FIG. 4D-4E show lusters of dysplastic stem cells (DSCs) differentiated cells or damaged cells in a superimposed UMAP plot (FIG. 4D) and their proportions in DMSO-treated and Pyr-treated Meta4 samples (FIG. 4E). FIG. 4F shows quantitative real time-PCR analysis showing relative expression of DSC markers, CD133 and CD166, in DMSO-treated vs Pyr-treated Meta4 organoids. FIG. 4G shows fluorescence-activated cell sorting analysis of the CD133 and CD166 positive cells in DMSO-treated and Pyr-treated Meta4 organoids. FIG. 4H shows the analysis of 7-AAD and annexin V expression in DMSO-treated and Pyr-treated Meta4 organoids. FIG. 4I shows a volcano plot of transcripts downregulated or upregulated in Pyr-treated vs DMSO-treated Meta4 cells. FIG. 4J-4K show STAT3-target upregulated (Fosb, Fos, and Jun) genes (FIG. 4J) and downregulated (Dusp4, Ddit3, and Areg) genes (FIG. 4K) in a superimposed UMAP plot.

FIG. 5A-5M show that pyrvinium treatment inhibits metaplasia progression to dysplasia in vivo. FIG. 5A schematically illustrates the experimental scheme of pyrvinium (Pyr) treatment in the Mist1-Kras mice at 3 months after Tamoxifen injection. FIG. 5B schematically illustrates the H&E (top) and immunostaining for UEA1 (middle) and Ki-67 (bottom) in the wild type mice treated with Pyr for 2 weeks and Mist1-Kras mice treated with DMSO or Pyr for 1 or 2 weeks. FIG. 5C schematically illustrates the quantitation of Ki-67-positive cells per 20× field. FIG. 5D shows panoramic view of immunostaining for H/K-ATPase in gastric corpus from wild type mice treated with Pyr for 2 weeks and Mist1-Kras mice treated with DMSO, Pyr for 1 week or 2 weeks. FIG. 5E shows quantitation of parietal cell-containing gastric units (GUs) per 100 GUs. FIG. 5F shows representative H&E images of gastric corpus from Mist1-Kras mice treated with DMSO or Pyr for 2 weeks. FIG. 5G shows percentages of dysplastic glands in remaining hyperplastic glands. FIG. 5H shows immunostaining for phospho-STAT3 (p-STAT3) and Pim-1 in gastric corpus from Mist1-Kras mice treated with DMSO or Pyr for 2 weeks. FIG. 5I-5J shows the results of co-immunostaining for CD4 and CD8 (FIG. 5I) and immunostaining for CD19 (FIG. 5J) in the gastric corpus from Mist1-Kras mice treated with DMSO or Pyr for 2 weeks. FIG. 5K-5L shows the co-immunostaining for CD3 and NK1.1 (FIG. 5K) and immunostaining for CD68 or CD163 (FIG. 5L) in the gastric corpus from Mist1-Kras mice treated with DMSO or Pyr for 2 weeks. FIG. 5M shows the quantitation of various immune cells per 20× field in Mist1-Kras mice treated with DMSO or Pyr. Mean±standard deviation. Unpaired t-test or one-way ANOVA with Tukey's multiple comparisons. ns, not significant. *P<0.05. **P<0.01. ***P<0.001. Scale bar=100 μM.

FIG. 6A-6H shows the pyrvinium targets human precancerous organoid (hPCO) with dysplastic features. FIG. 6A-6B show representative phase-contrast images of three pyrvinium (Pyr)-sensitive hPCO lines treated with DMSO, 1 μM of trametinib (Tra), 100 nM of pyrvinium and their combinations for 6 days (FIG. 6A) and quantitation of organoid diameters before and after treatment (FIG. 6B). FIG. 6C-6D show phase-contrast images of three Pyr-sensitive hPCO lines treated with DMSO, 1 μM of trametinib (Tra), 100 nM of pyrvinium and their combinations for 6 days (FIG. 6C) and quantitation of organoid diameters before and after treatment (FIG. 6D). FIG. 6E is a heatmap displaying Pyr response, morphology, and expression of TROP2, AQP5, and CD44v9 in twenty hPCO lines. FIG. 6F shows correlation graphs of Pyr response with morphology and TROP2 expression in hPCO lines. FIG. 6G shows bar graphs showing morphology or TROP2 expression scores between Pyr-sensitive and Pyr-resistant hPCO lines. FIG. 6H shows representative H&E images and co-immunostaining for CD44v9, AQP5, and TROP2 in three Pyr-sensitive and Pyr-resistant hPCO lines. Mean±standard deviation. Unpaired t-test or One-way ANOVA with Tukey's multiple comparisons. **P<0.01, ***P<0.001, ****P<0.0001. Scale bar=500 μM.

FIG. 7A-7L shows pyrvinium induces the cell death in dysplastic human precancerous (hPCO) organoids by blocking ERK and STAT3 signaling pathways. FIG. 7A-7B show Western blot analysis for phospho-STAT3 (p-STAT3) and phospho-ERK (p-ERK) in three pyrvinium (Pyr)-sensitive hPCO lines after Pyr treatment (FIG. 7A), and ratios of p-STAT3/total STAT3 (t-STAT3) and p-ERK/total ERK (t-ERK) (FIG. 7B). FIG. 7C-7D show Western blot analysis for p-STAT3 and p-ERK in three Pyr-resistant hPCO lines after Pyr treatment (FIG. 7C) and ratios of p-STAT3/t-STAT3 and p-ERK/t-ERK (FIG. 7D). FIG. 7E-7F show a Western blot for basal p-STAT3 and p-ERK levels in Pyr-sensitive and Pyr-resistant hPCO lines (FIG. 7E), and ratios of p-STAT3/t-STAT3 and p-ERK/t-ERK (FIG. 7F). FIG. 7G-7H show a Western blot for p-STAT3 and p-ERK in hPCO-3 after treatment of trametinib (Tra), Pyr, and their combinations (FIG. 7G), and ratios of p-STAT3/t-STAT3 and p-ERK/t-ERK (FIG. 7H). FIG. 7I shows representative immunostaining images of p-STAT3 in Pyr-sensitive or Pyr-resistant hPCO lines. FIG. 7J shows H&E and p-STAT3 immunostaining images of gastric tissues from which Pyr-sensitive or Pyr-resistant hPCO lines were derived. FIG. 7K-7L show phase-contrast images of Pyr-sensitive hPCO-2 treated with DMSO, Pyr, STAT3-IN-1, Sttatic, and Cryptotanshinone (CPT) for 6 days (FIG. 7K) and quantitation of organoid diameters before and after treatment (FIG. 7L). Mean±standard deviation. Unpaired t-test. *P<0.05, ****P<0.0001.

FIG. 8 is an annotated photograph showing a linear gastric mucosa strip (indicated by top dotted line) was obtained from a surgical specimen of gastric cancer patient. This section was adjacent to gastric cancer area (indicated by semicircular dotted line between clips). To prevent contamination from cancer cells, the area near the cancer lesion was marked with a wire (indicated by short, vertical arrow). The mucosa enclosed within the rectangular dotted box was utilized for the generation of gastric organoids.

FIG. 9 shows expression of Wnt/p-catein related genes upon treatment of MEK inhibitor, pyrvinium and CK1a inhibitor in dysplastic organoids. Real-time PCR analysis for Wnt-target genes including Axin2, Cdca4, Ephb3, Lrg5, Rnf43, Sp5, and Znrf3 in Meta4 organoids after treatment of DMSO vehicle, trametinib (Tra, 1 μM), pyrvinium (Pyr, 100 nM), and SSTC3 (1 μM) for 6 hours or 24 hours, respectively. Mean±SD. One-way ANOVA with Tukey's multiple comparisons.

FIG. 10A-10B show effects of STAT3 inhibitors in Meta3 organoids. FIG. 10A shows phase-contrast images of Meta3 organoids treated with either DMSO vehicle, 100 nM of pyrvinium (Pyr), or three STAT3 inhibitors, including STAT3-IN-1 (1 μM), Sttatic (2 μM), and Cryptotanshione (CPT, 5 μM) for 3 days. FIG. 10B shows quantitation of organoid diameters before and after treatment. Mean±SO. Two-way ANOVA with Tukey test for pairwise comparisons. Scale bar=500 μm.

FIG. 11 is a bar graph showing expression of STAT3-target genes upon treatment of pyrvinium (Pyr) in dysplastic organoids. Real-time PCR analysis for 15 STAT3-target genes in Meta4 organoids after treatment of pyrvinium (100 nM) for 48 hours. Three replicates were performed. Two-tailed Mann-Whitney test.

FIG. 12 shows images depicting normal tissues in multiple organs, including the stomach, esophagus, liver, kidney, heart, lung, and spleen were unaffected by pyrvinium (Pyr) treatment in vivo. Hematoxylin and eosin images were obtained from wild-type mice treated with either dimethyl sulfoxide (DMSO) or Pyr (4 mg/L in drinking water) for a duration of 2 weeks. Scale bar=100 μm.

FIG. 13A-13B show alterations in regulatory T cells by pyrvinium treatment in Mist1-Kras mice. FIG. 13A shows immunostaining for CD3 and CD25 in the gastric corpus from Mist1-Kras mice treated with DMSO or Pyr for 2 weeks. FIG. 13B shows quantitation of various immune cells per 20× field and ratio of regulatory T cells (Treg) per total T cells in the wild type mice and Mist1-Kras mice treated with DMSO or Pyr. Mean±SD. Two-tailed Mann-Whitney test. Scale bar=100 μm.

FIG. 14 shows gene expression analysis for Mist1-Kras mice at 3 months after Tamoxifen injection were administered either DMSO or pyrvinium (Pyr) for 2 weeks. Quantitative real time-PCR analysis was performed for myeloid derived suppressor cell markers, including Slfn4, Nos2, and Arg1 and Itgam1. Relative quantification of each gene expression level was normalized to Gapdh gene expression. Mean±standard deviation. Two-tailed Mann-Whitney test.

FIG. 15A-15C shows the effects of pyrvinium (Pyr) treatment in fourteen various human precancerous organoid (hPCO) lines established from human stomach samples of gastric cancer patients. H&E, co-immunostaining for CD44v9, Aqp5 and Trop2, and phase-contrast images in two Pyr-sensitive (FIG. 15A), six Pyr-moderate (FIG. 15B), and six Pyr-resistant (FIG. 15C) hPCO lines treated with DMSO, 1 μM of trametinib (Tra), 100 nM of Pyr and their combinations for 6 days. Scale bar=100 μm.

FIG. 16 shows images depicting cell death induced by pyrvinium (Pyr) in human precancerous organoid (hPCO) line. Live/dead cell analysis using double staining of Calcein AM and Ethidium Homodimer-1 (EthD-1) after DMSO, trametinib (Tra, 1 μM), pyrvinium (Pyr, 100 nM), and combination of Tra and Pyr in Pyr-sensitive hPCO-34 line and percentages of live and dead cells after treatment. Scale bar=500 μm.

FIG. 17A-17B show the effects of Casein Kinase 1a (CK1a) activator treatment in human precancerous organoid (hPCO) line. FIG. 17A shows phase-contrast images of pyrvinium-sensitive hPCO-34 treated with DMSO vehicle or varying concentrations (0.1, 0.2, 0.5, and 1.0 μM) of SSTC3, a specific CK1a activator, for 6 days. FIG. 17B shows quantitation of organoid diameters before and after treatment. Mean±SD. Two-way ANOVA (with Tukey test for pairwise comparisons). Scale bar=500 μm.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “-” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, the terms “salt” or “salts” refers to an acid addition or base addition salt of a compound of the invention. “Salts” include in particular “pharmaceutical acceptable salts.” The term “pharmaceutically acceptable salts” refers to salts that retain the biological effectiveness and properties of the compounds of this invention and, which typically are not biologically or otherwise undesirable.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the terms “salt” or “salts” refers to an acid addition or base addition salt of a compound of the disclosure. “Salts” include in particular “pharmaceutical acceptable salts.” The term “pharmaceutically acceptable salts” refers to salts that retain the biological effectiveness and properties of the compounds of this disclosure and, which typically are not biologically or otherwise undesirable. In many cases, the compounds described herein may form acid and/or base salts by virtue of the presence of amino and/or carboxyl groups or groups similar thereto.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆ alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkoxyalkyl,” as used herein, refers to an alkoxy group, as defined herein, appended to the parent molecular moiety through an alkyl group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “hydroxyl” or “hydroxy,” as used herein, means an —OH group.

Terms such as “alkyl,” “alkylene,” etc. may be preceded by a designation indicating the number of atoms present in the group in a particular instance (e.g., “C₁₋₄alkyl,” or “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, hydroxy, alkoxy, alkoxyalkyl, alkylene, amino, alkylamino, acylamino, aminoalkyl, sulfonylamino, sulfinylamino, sulfonyl, alkylsulfonyl, arylsulfonyl, aminosulfonyl, sulfinyl, —COOH, ketone, amide, carbamate, and acyl.

Terms such as “alkyl,” “alkylene,” etc. may be preceded by a designation indicating the number of atoms present in the group in a particular instance (e.g., “C₁₋₄alkyl,” “C₁₋₄alkylene”).

These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups.

Described herein is a method of reprogramming a pre-cancerous mucosa to a non-cancerous state, the method comprising identifying a pre-cancerous mucosa comprising a metaplastic cell or a dysplastic cell; and contacting the metaplastic cell or the dysplastic cell with a compound of formula (I), or a salt thereof:

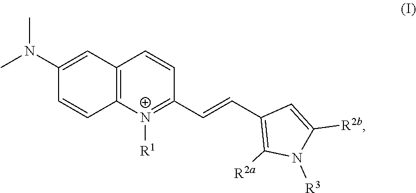

wherein:
R¹ is C₁₋₆alkyl;
R^{2a} and R^{2b} are each independently C₁₋₆alkyl; and
R³ is C₆₋₁₂aryl, wherein R³ is optionally substituted with 1-5 R^{X}
R^{X}, at each occurrence, is independently C₁₋₆alkyl, C₁₋₄haloalkyl, halogen, cyano, —N(R^{Xa})₂, —OR^{Xb}, —SR^{Xb}, —C(O)R^{Xb}, —CO₂R^{Xb}, —C(O)N(R^{Xa})₂, —SO₂R^{Xa}, -L²-Y², —O-L^{X}-Y^{X}, —S-L^{X}-Y^{X}, or —N(R^{Xa})-L^{X}-Y^{X};
L^{X}, at each occurrence, is independently C₁₋₆alkylene, C₂₋₆alkenylene, or C₂₋₆alkynylene;
Y^{X}, at each occurrence, is independently hydrogen, cyano, halogen, haloalkyl, —OH, —N(R^{Xa})₂, —OR^{Xb}, —SR^{Xb}, —C(O)R^{Xb}, —CO₂R^{Xb}, —C(O)N(R^{Xa})₂, or —SO₂R^{Xa};
R^{Xa}, at each occurrence, is independently hydrogen, C₁₋₄alkyl, or —C(O)C₁₋₄alkyl; and
R^{Xb}, at each occurrence, is independently hydrogen, C₁₋₄alkyl, C₁₋₂haloalkyl, or —C(O)C₁₋₄alkyl.

The metaplastic cell or dysplastic cell may be located within a living organism, e.g., a subject such as a non-human animal or a human. As used herein, the term “living organism” means any biological living system that functions as an individual life form. The dysplastic cell may be positive for CD133, CD166, and Trop2 expression. The dysplastic cell may be a dysplastic gastric cell, a dysplastic esophageal cell, a dysplastic pancreatic cell, a dysplastic colon cell, or dysplastic ovarian cell. The dysplastic gastric cell may be part of an organoid. The metaplastic gastric cell may be positive for CD133, CD166, and Trop2 expression. The metaplastic cell may be a metaplastic gastric cell, a metaplastic esophageal cell, a metaplastic pancreatic cell, a metaplastic colon cell, or metaplastic ovarian cell. The metaplastic gastric cell may be part of an organoid.

In another aspect, described herein is a method of reducing the risk of cancer development in a living organism, the living organism containing a metaplastic cell or a dysplastic cell, the method comprising administering to the living organism the compound of formula (I) or a salt thereof. The method may further comprise administering a second agent to the living organism. The second agent may be a kinase inhibitor, such as a MEK inhibitor, for example trametinib, binimetinib, cobimetinib, and/or selumetinib.

These and other aspects of the disclosure are set out in detail below.

### Compounds

Compounds of the present disclosure may include a compound of formula (I), or a salt thereof:

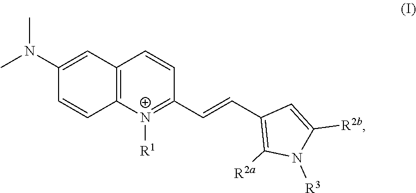

wherein:
R¹ is C₁₋₆alkyl;
R^{2a} and R^{2b} are each independently C₁₋₆alkyl; and
R³ is C₆₋₁₂aryl, wherein R³ is optionally substituted with 1-5 R^{X}
R^{X}, at each occurrence, is independently C₁₋₆alkyl, C₁₋₄haloalkyl, halogen, cyano, —N(R^{Xa})₂, —OR^{Xb}, —SR^{Xb}, —C(O)R^{Xb}, —CO₂R^{Xb}, —C(O)N(R^{Xa})₂, —SO₂R^{Xa}, -L²-Y², —O-L^{X}-Y^{X}, —S-L^{X}-Y^{X}, or —N(R^{Xa})-L^{X}-Y^{X};
L^{X}, at each occurrence, is independently C₁₋₆alkylene, C₂₋₆alkenylene, or C₂₋₆alkynylene;
Y^{X}, at each occurrence, is independently hydrogen, cyano, halogen, haloalkyl, —OH, —N(R^{Xa})₂, —OR^{Xb}, —SR^{Xb}, —C(O)R^{Xb}, —CO₂R^{Xb}, —C(O)N(R^{Xa})₂, or —SO₂R^{Xa};
R^{Xa}, at each occurrence, is independently hydrogen, C₁₋₄alkyl, or —C(O)C₁₋₄alkyl; and
   R^{Xb}, at each occurrence, is independently hydrogen, C₁₋₄alkyl, C₁₋₂haloalkyl, or —C(O)C₁₋₄ alkyl.

In some instances, R¹ may be methyl. In some instances, R^{2a} and R^{2b} may each be methyl. In some instances, R³ may be unsubstituted phenyl. In some instances, the compound of formula (I) may be pyrvinium, shown below, or a salt thereof.

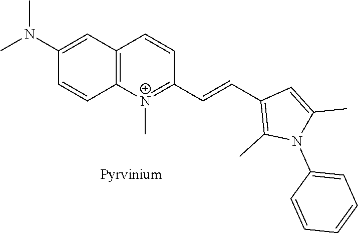 [SMILES: Cc1cc(/C=C/c2ccc3cc(N(C)C)ccc3[n+]2C)c(C)n1-c1ccccc1]

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

It should be understood that the compound may possess tautomeric forms, as well as geometric isomers, and that these also constitute an aspect of the invention.

In the compounds of formula (I), and any subformulas, any “hydrogen” or “H,” whether explicitly recited or implicit in the structure, encompasses hydrogen isotopes ¹H (protium) and ²H (deuterium).

### Salts

In various instances, the compound of formula (I) may be present as salt. The salt of the compound of formula (I) may be a pharmaceutically acceptable salt. As used herein, the term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts may be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

In some instances, the salt of the compound of formula (I) may be a salt of formula (I-A),

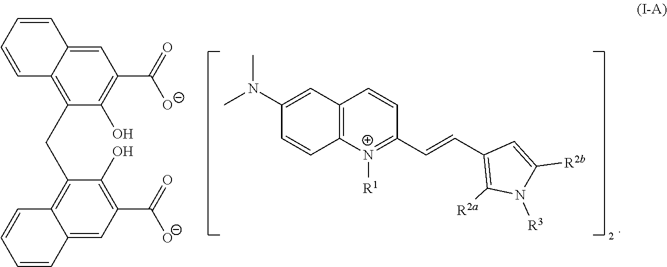

In some instances, the salt of formula (I-A) may be pyrvinium pamoate, shown below.

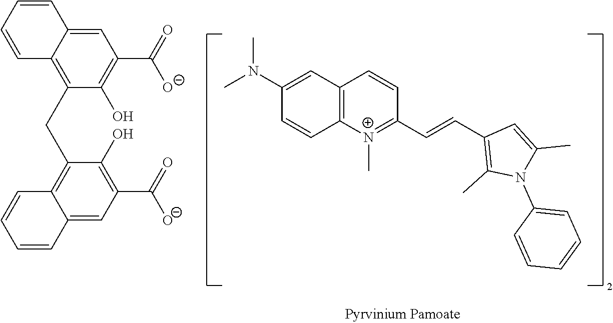

### Formulations and Routes for Administration

The disclosed compounds may be incorporated into pharmaceutical compositions suitable for administration to living organism, e.g., a subject such as a patient, which may be a human or non-human.

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Where clinical applications are contemplated, it will be necessary to prepare pharmaceutical compositions comprising the compound of formula (I), or a salt thereof, or any additional therapeutic agent disclosed herein in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers may also be employed when recombinant cells are introduced into a patient. Aqueous compositions of the present disclosure in an effective amount may be dissolved or dispersed in a pharmaceutically acceptable carrier or aqueous medium. Such compositions may also be referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like.

Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions. Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%. Suitable colorants include a colorant such as an FD&C dye. When used, the amount of colorant in a systemic or topical composition is typically about 0.005 to about 0.1%. Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%. Suitable antioxidants include butylated hydroxyanisole (“BHA”), butylated hydroxytoluene (“BHT”), and vitamin E. The amount of antioxidant(s) in a systemic or topical composition is typically about 0.1 to about 5%. Suitable preservatives include benzalkonium chloride, methyl paraben and sodium benzoate. The amount of preservative(s) in a systemic or topical composition is typically about 0.01 to about 5%. Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%. Suitable suspending agents include AVICEL RC-591 (from FMC Corporation of Philadelphia, PA) and sodium alginate.

The amount of suspending agent(s) in a systemic or topical composition is typically about 1 to about 8%. Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active (e.g., compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention. Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols. The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%. The amount of thickener(s) in a topical composition is typically about 0% to about 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%. The amount of fragrance in a topical composition is typically about 0% to about 0.5%, particularly, about 0.001% to about 0.1%. Suitable pH adjusting additives include HCl or NaOH in amounts sufficient to adjust the pH of a topical pharmaceutical composition.

### Therapeutically Effective Amounts of Compositions

An effective amount of the therapeutic agent(s) of the present disclosure may be determined based on the intended goal, for example (i) inhibition of tumor cell proliferation or (ii) elimination of tumor cells. The term “unit dose” refers to physically discrete units suitable for use in a subject, each unit containing a predetermined quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and may be specific to the individual.

In some instances, the dose range of the compound of formula (I), or salt thereof, may be about 0.5 mg/kg body weight to about 500 mg/kg body weight. The term “body weight” is applicable when an animal is being treated. When isolated cells are being treated, “body weight,” as used herein, means “total cell weight”. The term “total weight” may be used to apply to both isolated cell and animal treatment. All concentrations and treatment levels expressed as “body weight” or “kg” in this application may also be considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that various dosage levels may be used, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Any of the above dosage ranges or dosage levels may be employed for the compound of formula (I), or salt thereof, in combination with a second therapeutic agent.

As is understood in the art, a specific dose level of active compounds such as the compound of formula (I), or a salt thereof, alone, or in combination with a second therapeutic agent, for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In some implementations, the compound of formula (I), or a salt thereof, may be administered alone, or in combination with a second therapeutic agent. When a second therapeutic agent is co-administered with the compound of formula (I) or salt thereof, if the second therapeutic agent dose does not exceed previously quoted toxicity levels, the effective amounts of the second therapeutic agents may be defined as those amounts effective to reduce the pre-cancer growth. The amount effective to reduce the pre-cancer growth may be determined by monitoring the animal or patient and measuring those physical and biochemical parameters of health and disease that are indicative of the success of a given treatment. Such methods are routine in animal testing and clinical practice.

In some implementations, chemotherapy may be administered, as is typical, in regular cycles. A cycle may involve one dose, after which several days or weeks without treatment ensues for normal tissues to recover from the drug's side effects. Doses may be given several days in a row followed by a period of rest. If more than one drug is used, the treatment plan will specify how often and exactly when each drug should be given. The number of cycles a person receives may be determined before treatment starts (based on the type and stage of pre-cancer) or may be flexible, to consider how quickly mucosal recovery is observed. Certain serious side effects may also require doctors to adjust chemotherapy plans to allow the patient time to recover.

### Combination Therapy

In some instances, the compound of formula (I), or a salt thereof may be used in combination with a second therapeutic agent. Additional therapeutic agents contemplated for use in combination with the compound of formula (I), or a salt thereof, may include, but are not limited to, anticancer agents. Anticancer agents may include, but are not limited to, radiotherapy, chemotherapy, gene therapy, hormonal therapy or immunotherapy that targets cancer/tumor cells.

To kill cells, induce cell-cycle arrest, inhibit cell growth, inhibit metastasis, inhibit angiogenesis or otherwise reverse or reduce the malignant phenotype of pre-cancer cells, using the methods and compositions of the present disclosure, one would generally contact a cell with the compound of formula (I), or a salt thereof, in combination with a second therapeutic agent. These compositions would be provided in a combined amount effective to kill or inhibit proliferation of the cell. This process may involve contacting the cells with the compound of formula (I), or a salt thereof, in combination with a second therapeutic agent or factor(s) at the same time. This may be achieved by contacting the cell with a single composition or pharmacological formulation that includes both agents, or by contacting the cell with two distinct compositions or formulations, at the same time, wherein one composition includes the compound of formula (I), or a salt thereof, and the other includes the second agent.

Alternatively, treatment with the compound of formula (I), or a salt thereof, may precede or follow the additional agent treatment by intervals ranging from minutes to weeks. In embodiments where the second agent is applied separately to the cell, one would generally ensure that a marked period of time did not expire between the time of each delivery, such that the agent would still be able to exert an advantageously combined effect on the cell. In such instances, it is contemplated that one would contact the cell with both modalities within about 12-24 hr of each other, or, alternatively within about 6-12 hr of each other. In some situations, it may be desirable to extend the time period for treatment markedly, however, where several days (2, 3, 4, 5, 6 or 7) to several weeks (1, 2, 3, 4, 5, 6, 7 or 8) lapse between the respective administrations.

It also is conceivable that more than one administration of either pyrvinium or a salt thereof thereof in combination with a second therapy such as an anticancer agent or therapy will be desired. Various combinations may be employed, where pyrvinium or a salt thereof is “A” and the second therapeutic agent is “B,” as exemplified below:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations may be contemplated. Again, to achieve cell killing by inducing apoptosis, both agents may be delivered to a cell in a combined amount effective to kill the cell.

### Chemotherapeutic Agents

In some implementations, the chemotherapeutic agents may be used in combination with the compound of formula (I), or a salt thereof, to treat pre-cancer. Examples of such chemotherapeutic agents include, but are not limited to, kinase inhibitors. Examples of kinase inhibitors include MEK inhibitors, such as trametinib, binimetinib, cobimetinib, and/or selumetinib.

### Immunotherapeutic Agents

Immunotherapeutics may also be employed in the present disclosure in combination with the compound of formula (I) or a salt thereof in treating pre-cancer. Generally, immunotherapeutics rely on the use of immune effector cells and molecules to target and destroy pre-cancer cells. The immune effector may be, for example, an antibody specific for some marker on the surface of a tumor cell. The antibody alone may serve as an effector of therapy, or it may recruit other cells to effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on most other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

Checkpoint inhibitor therapy is another form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function. Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD-1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies that bind to either PD-1 or PD-L1 and therefore block the interaction may allow the T-cells to attack the tumor.

### Other Agents

It is contemplated that other agents may be used in combination with the compound of formula (I), or the salt thereof, to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present disclosure by establishing an autocrine or paracrine effect on hyperproliferative cells. Increased intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyperproliferative efficacy of the treatments. Inhibitors of cell adhesion may be contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the compositions described herein to improve the treatment efficacy.

### Methods of Treatment

To kill cells, induce cell cycle arrest, inhibit cell growth, inhibit metastasis, inhibit angiogenesis, or otherwise reverse or reduce the malignant phenotype of pre-cancer cells, or reduce the risk of developing cancer, a cell, e.g., a metaplastic cell or a dysplastic cell, may be contacted with the compound of formula (I), or a salt thereof. The terms “contacted” and “exposed,” when applied to a cell, are used herein to describe the process by which a therapeutic agent is delivered to a target cell or are placed in direct juxtaposition with the target cell. To achieve cell killing or stasis, the therapeutic agent is delivered to a cell in an amount effective to induce cell cycle arrest, inhibit cell growth and induce apoptosis in the cell.

In some instances, methods described herein may reprogram a pre-cancerous mucosa to a non-cancerous state, wherein the pre-cancer mucosa contains a metaplastic cell or a dysplastic cell. Example methods for reprogramming a pre-cancerous mucosa to a non-cancerous state may comprise identifying a pre-cancerous mucosa comprising a metaplastic cell or a dysplastic cell; and contacting the metaplastic cell or the dysplastic cell with a compound of formula (I), or a salt thereof. The metaplastic cell or the dysplastic cell may be located within a living organism. The pre-cancerous mucosa may comprise a dysplastic cell. The method may comprise killing the dysplastic cell. The dysplastic cell may be positive for CD133 expression, CD166 expression, and/or Trop2 expression. The dysplastic cell may be a dysplastic gastric cell, a dysplastic esophageal cell, a dysplastic pancreatic cell, a dysplastic colon cell, or dysplastic ovarian cell. The dysplastic gastric cell may be part of an organoid. The pre-cancerous mucosa may comprise a metaplastic cell. The metaplastic gastric cell may be positive for CD133, CD166, and/or Trop2 expression. The metaplastic cell may be a metaplastic gastric cell, a metaplastic esophageal cell, a metaplastic pancreatic cell, a metaplastic colon cell, or a metaplastic ovarian cell. The metaplastic gastric cell may be part of an organoid.

In some instances, the methods described herein may reduce the risk of cancer in a living organism e.g., a subject such as a non-human animal or a human, containing a metaplastic cell or a dysplastic cell. Example methods for reducing the risk of cancer development in the living organism containing the metaplastic cell or the dysplastic cell may comprise administering to the living organism a compound of formula (I), or a salt thereof. The method may inhibit STAT3 and MEK/ERK signaling pathways in the metaplastic cell or the dysplastic cell. In some instances, the living organism may be a non-human animal. In other instances, the living organism is a human. The human may be at risk for developing cancer. The method may further comprise administering a second agent to the living organism. The second agent may comprise a kinase inhibitor. The kinase inhibitor may comprise a MEK inhibitor. The MEK inhibitor may comprise trametinib, binimetinib, cobimetinib, and/or selumetinib.

The compound of formula (I), a salt thereof, or a pharmaceutical composition comprising the same, may be administered to a subject once or more than once and at intervals ranging from minutes to weeks. Generally, a period of time should not expire between the same, may be administered as a therapeutic agent time of each delivery that would prevent the agent from being able to exert an advantageous effect on the cell. However, in some situations, it may be desirable to extend the time period for treatment where several days (2, 3, 4, 5, 6 or 7) to several weeks (1, 2, 3, 4, 5, 6, 7 or 8) lapse between the respective administrations.

Administration of the compound of formula (I) or a salt thereof to a subject may be by any method known in the art for delivery of a therapeutic agent to a subject. For example, such methods may include, but are not limited to, oral, nasal, intramuscular, or intraperitoneal administration. Methods of administration are disclosed in detail elsewhere in this application.

### Metaplasia and Dysplasia

Metaplasia is the transformation of one differentiated cell type to another differentiated cell type. The change from one type of cell to another may be part of a normal maturation process or may be caused by some sort of abnormal stimulus. In simplistic terms, it is as if the original cells are not robust enough to withstand their environment, so they transform into another cell type better suited to their environment. If the stimulus causing metaplasia is removed or ceases, tissues return to their normal pattern of differentiation. Metaplasia is not synonymous with dysplasia and is not considered to be an actual cancer. Today, metaplastic changes are usually considered to be an early phase of carcinogenesis, specifically for those with a history of cancers or who are known to be susceptible to carcinogenic changes. Metaplastic change is thus often viewed as a premalignant condition.

Dysplasia is any of various types of abnormal growth or development of cells (microscopic scale) or tissues (macroscopic scale), and the abnormal histology or anatomical structure(s) resulting from such growth.

In one of the modern histopathological senses of the term, dysplasia may be differentiated from other categories of tissue change including hyperplasia, metaplasia, and neoplasia. Dysplasia is generally considered pre-cancerous.

Thus, metaplasia describes the transformation of one mature type of cell into another mature type of cell, while dysplasia describes an increased amount of immature cell types, often abnormal. Both metaplasia and dysplasia typically result from chronic environmental stressors.

### Cancers to be Treated According to the Methods

A compound of formula (I), or a salt thereof, is shown here to inhibit metaplastic and dysplastic cells and therefore may be useful to treat diseases of uncontrolled proliferation, in particular pre-cancers. Thus, the compound of formula (I) or a salt thereof may be used as a therapeutic agent for treating pre-cancer in a subject, such as hepatocellular carcinoma, Barrett's esophageal, bladder, breast, gastric, colon, head & neck, lung cell, mesothelioma, and/or cervical pre-cancers. More specifically, in some instances, the pre-cancer cells may be positive for CD133, CD166, and/or Trop2 expression.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

All the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

Various embodiments and aspects of the inventions described herein are summarized by the following clauses:
Clause 1. A method for reprogramming a pre-cancerous mucosa to a non-cancerous state, the method comprising:
   identifying a pre-cancerous mucosa comprising a metaplastic cell or a dysplastic cell; and
   contacting the metaplastic cell or the dysplastic cell with a compound of formula (I), or a salt thereof:

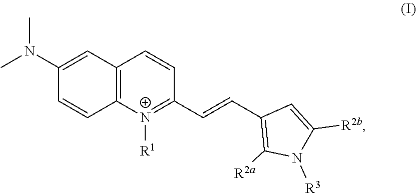

wherein:
R¹ is C₁₋₆alkyl;
R^{2a} and R^{2b} are each independently C₁₋₆alkyl; and
R³ is C₆₋₁₂aryl, wherein R³ is optionally substituted with 1-5 R^{X}
R^{X}, at each occurrence, is independently C₁₋₆alkyl, C₁₋₄haloalkyl, halogen, cyano, —N(R^{Xa})₂,
   —OR^{Xb}, —SR^{Xb}, —C(O)R^{Xb}, —CO₂R^{Xb}, —C(O)N(R^{Xa})₂, —SO₂R^{Xa}, -L²-Y², —O-L^{X}-Y^{X}, —S-L^{X}-Y^{X}, or —N(R^{Xa})-L^{X}-Y^{X};
L^{X}, at each occurrence, is independently C₁₋₆alkylene, C₂₋₆alkenylene, or C₂₋₆alkynylene;
Y^{X}, at each occurrence, is independently hydrogen, cyano, halogen, haloalkyl, —OH, —N(R^{Xa})₂,
   —OR^{Xb}, —SR^{Xb}, —C(O)R^{Xb}, —CO₂R^{Xb}, —C(O)N(R^{Xa})₂, or —SO₂R^{Xa};
R^{Xa}, at each occurrence, is independently hydrogen, C₁₋₄alkyl, or —C(O)C₁₋₄alkyl; and
R^{Xb}, at each occurrence, is independently hydrogen, C₁₋₄alkyl, C₁₋₂haloalkyl, or —C(O)C₁₋₄alkyl.
Clause 2. The method of clause 1, wherein the metaplastic cell or the dysplastic cell is contacted with a salt of the compound of formula (I).
Clause 3. The method of clause 2, wherein the salt of the compound of formula (I) is a salt of formula (I-A),

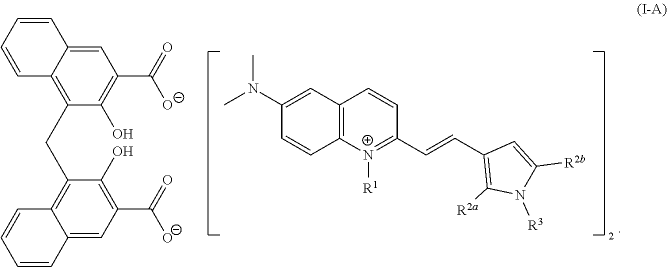

Clause 4. The method of any one of clauses 1-3, wherein R¹ is methyl.
Clause 5. The method of any one of clauses 1-4, wherein R^{2a} and R^{2b} are each methyl.
Clause 6. The method of any one of clauses 1-5, wherein R³ is unsubstituted phenyl.
Clause 7. The method of any one of clauses 1-6, wherein the compound of formula (I) is pyrvinium.
Clause 8. The method of any one of clauses 1-7, wherein the metaplastic cell or the dysplastic cell is located within a living organism.
Clause 9. The method of any one of clauses 1-8, wherein the pre-cancerous mucosa comprises a dysplastic cell.
Clause 10. The method of clause 9, wherein the method comprises killing the dysplastic cell.
Clause 11. The method of clause 9 or 10, wherein the dysplastic cell is positive for CD133 expression, CD166 expression, and/or Trop2 expression.
Clause 12. The method of any one of clauses 9-11, wherein the dysplastic cell is a dysplastic gastric cell, a dysplastic esophageal cell, a dysplastic pancreatic cell, a dysplastic colon cell, or a dysplastic ovarian cell.
Clause 13. The method of clause 12, wherein the dysplastic gastric cell is part of an organoid.
Clause 14. The method of any one of clauses 1-13, wherein the pre-cancerous mucosa comprises a metaplastic cell.
Clause 15. The method of clause 14, wherein the metaplastic gastric cell is positive for CD133, CD166, and/or Trop2 expression.
Clause 16. The method of any one of clauses 1-15, wherein the metaplastic cell is a metaplastic gastric cell, a metaplastic esophageal cell, a metaplastic pancreatic cell, a metaplastic colon cell, or a metaplastic ovarian cell.
Clause 17. The method of clause 16, wherein the metaplastic gastric cell is part of an organoid.
Clause 18. A method for reducing the risk of cancer development in a living organism containing a metaplastic cell or a dysplastic cell, the method comprising:
   administering to the living organism a compound of formula (I), or a salt thereof:

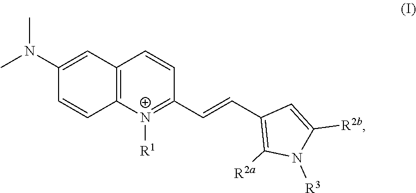

wherein:
R¹ is C₁₋₆alkyl;
R^{2a} and R^{2b} are each independently C₁₋₆alkyl; and
R³ is C₆₋₁₂aryl, wherein R³ is optionally substituted with 1-5 R^{X}
R^{X}, at each occurrence, is independently C₁₋₆alkyl, C₁₋₄haloalkyl, halogen, cyano, —N(R^{Xa})₂,
   —OR^{Xb}, —SR^{Xb}, —C(O)R^{Xb}, —CO₂R^{Xb}, —C(O)N(R^{Xa})₂, —SO₂R^{Xa}, -L²-Y², —O-L^{X}-Y^{X}, —S-L^{X}-Y^{X}, or —N(R^{Xa})-L^{X}-Y^{X};
L^{X}, at each occurrence, is independently C₁₋₆alkylene, C₂₋₆alkenylene, or C₂₋₆alkynylene;
Y^{X}, at each occurrence, is independently hydrogen, cyano, halogen, haloalkyl, —OH, —N(R^{Xa})₂,
   —OR^{Xb}, —SR^{Xb}, —C(O)R^{Xb}, —CO₂R^{Xb}, —C(O)N(R^{Xa})₂, or —SO₂R^{Xa};
R^{Xa}, at each occurrence, is independently hydrogen, C₁₋₄alkyl, or —C(O)C₁₋₄alkyl; and
   R^{Xb}, at each occurrence, is independently hydrogen, C₁₋₄alkyl, C₁₋₂haloalkyl, or —C(O)C₁₋₄alkyl.
Clause 19. The method of clause 18, wherein a salt of the compound of formula (I) is administered to the living organism.
Clause 20. The method of clause 18 or 19, wherein the salt of the compound of formula (I) is a salt of formula (I-A),

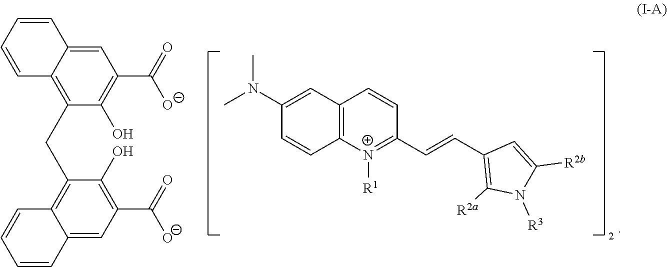

Clause 21. The method of any one of clauses 18-20, wherein the compound of formula (I) is pyrvinium.
Clause 22. The method of any one of clauses 18-21, wherein the method inhibits STAT3 and MEK/ERK signaling pathways in the metaplastic cell or the dysplastic cell.
Clause 23. The method of any one of clauses 18-22, wherein the living organism is a non-human animal.
Clause 24. The method of any one of clauses 18-23, wherein the living organism is a human.
Clause 25. The method of clause 24, wherein human is at risk for developing cancer.
Clause 26. The method of any one of clauses 18-25, the method further comprising administering a second agent to the living organism.
Clause 27. The method of clause 26, wherein the second agent comprises a kinase inhibitor.
Clause 28. The method of clause 27, wherein the kinase inhibitor comprises a MEK inhibitor.
Clause 29. The method of clause 28, wherein the MEK inhibitor comprises trametinib, binimetinib, cobimetinib, and/or selumetinib.
Clause 30. Use of pyrvinium or a salt thereof in the manufacture of a medicament for reprograming a pre-cancerous mucosa to a non-cancerous state or reducing the risk of cancer development.

### Examples

### Materials and Methods

### Organoid Establishment, Culture and Drug Treatment

To establish human pre-cancer organoids (hPCO), fresh tissues were obtained from the gastric cancer patients who underwent curative gastrectomy at SNUH (IRB No. H-1806-166-954). Organoid lines were established as described in a previous study.

Meta3, Meta4, and hPCO organoids were cultured in Matrigel (ECM, Sigma) with Mouse or Human IntestiCult medium (StemCell Technology) supplemented with 1% of penicillin/streptomycin (Gibco) in 48-well plates and medium was replaced every 3 days. The organoids were split every 5-7 days before they formed budding structures.

Trametinib, pyrvinium pamoate, SSTC3 and three STAT3 inhibitors (STAT3-IN-1, Sttatic, Cryptotanshinone) (all from MedChemExpress) were dissolved in DMSO. Mouse or human organoids were split and cultured in the Mouse or Human IntestiCult media for 1-2 days until they formed three-dimensional (3D) spherical structures, then the media was switched to media containing either DMSO vehicle or a certain concentration of each drug and cultured for 3 days. The EVOS M7000 inverted microscope were used to obtain phase contrast images of organoids. All experiments were repeated at least three times.

### Immunostaining

To prepare formaldehyde-fixed paraffin-embedded (FFPE) organoids/spheres, Matrigel domes containing organoids/spheres were fixed in 4% PFA at room temperature for 30 min. The fixed organoids were embedded in HistoGel™ (Thermo Fisher Scientific) followed by a wash in PBS for 1 min and processed according to a standard histological protocol for paraffin embedding. Human tissues were fixed in 10% neutral buffered formalin (NBF) for 1 week at room temperature and mouse tumor tissues were fixed in 4% paraformaldehyde (PFA) solution overnight at 4° C. The fixed tissues were transferred into 70% ethanol and processed according to a standard histological protocol for subsequent paraffin embedding. Four to five micrometers of organoid/sphere or tissue paraffin sections were de-paraffinized in Histoclear solution (Electron Microscopy Services) and rehydrated through a series of ethanol (100%, 95% and 75%).

Antigen retrieval was performed using a pH 6 or pH 9 target retrieval solution (Dako) in a pressure cooker for 15 min and cooled for 1 hour. For immunofluorescence staining, each section was incubated in serum-free protein block solution (Dako) at room temperature for 1.5 hours. Primary antibodies were diluted in Antibody Diluent with background reducing components (Dako) and applied to sections, then incubated overnight at 4° C. Slides were washed in PBS for 5 min three times and secondary antibodies diluted in Antibody Diluent (Dako) were applied to each section. Sections were incubated at room temperature for 1 hour and PBS with Hoechst (1:5000) was applied to the sections for 5 min for nuclei counterstaining. Slides were washed in PBS for 5 min three times, mounted with ProLong™ Gold Antifade Mountant (ThermoFisher) and cover slipped. All fluorescence images were acquired using a Zeiss Axio Imager M2 microscope with Axiovision digital imaging system or scanned using the Aperio Versa 200 Fluorescent Slide Scanner (Leica) at the Vanderbilt Digital Histology Shared Resource. Preparation and overlay of fluorescence images were performed using Adobe Photoshop.

For immunohistochemical staining, sections were blocked in Peroxidase Blocking Solution (Vector Laboratories) at room temperature for 20 min followed by serum-free protein block solution (Dako) at room temperature for 1.5 hours. Primary antibodies were diluted in Antibody Diluent with background reducing components (Dako) and applied to sections, then incubated overnight at 4° C. Horseradish-peroxidase (HRP)-conjugated secondary antibodies were applied to sections and incubated for 15 min at room temperature followed by three washes in PBS for 5 min each. ImmPRESS polymer detection reagent and ImmPACT DAB substrate kits (Vector Laboratories) were used for antibody detection. Images were scanned on a SCN400 slide scanner (Leica) at 20× magnification.

### Quantitative Real-Time PCR (RT-PCR)

Total RNAs were extracted from Meta3 or Meta4 organoids using the Trizol (Invitrogen) reagent. cDNAs were synthesized using iScript gDNA clear cDNA synthesis kit (Bio-Rad) and RT-PCR was performed using SsoAdvanced™ Universal SYBR Green supermix (Bio-Rad) and a CFX96 Real-Time PCR Detection System (Bio-Rad). GAPDH, a house keeping gene, was used to normalize gene expression levels. All reactions were performed in triplicate and relative gene expression levels were obtained by the comparative ΔΔCt method using CFX Maestro software (Bio-Rad).

### Western Blot

Total proteins were extracted from organoids using radioimmunoprecipitation assay buffer (10 mmol/L Tris-HC, pH 7.2, 150 mmol/L NaCl, 5 mmol/L EDTA, 0.1% sodium dodecyl sulfate, 1.0% Triton X-100, 1% deoxycholate) with protease inhibitors. The protein concentration was measured The Bio-Rad Protein Assay (Bio-Rad Laboratories, Hercules, CA). 5 to 10 μg of total protein was loaded onto 12.5% or 10% sodium dodecyl sulfate/polyacrylamide gel electrophoresis (PAGE) and transferred onto polyvinylidene difluoride membranes (Millipore, Billerica, MA). The membranes were blocked with 5% skim milk in 1×TBS (Corning) with 0.1% Tween 20 (Bio-Rad) for 1 hour at room temperature and incubated overnight at 4° C. with primary antibodies diluted in 5% skim milk in TBS-T. The following day, the membranes were washed three times with TBS-T and incubated with HRP conjugated mouse secondary antibody (Promega) diluted in 5% skim milk in TBS-T for 1 hour at room temperature. After washes with TBS-T, chemiluminescence was detected with an Amersham Imager 680 instrument (GE Healthcare) after 1 mL of the SuperSignal West Femto Maximum Sensitivity Substrate (ThermoFisher) was applied to the membranes.

### Quantification and Statistical Analysis

The number and diameters of mouse and human organoids treated with drugs were manually counted and measured at day 0 and 3 or 6 after treatment from four entire well images of each condition at 4× magnification. For counting live or dead cells, organoids were stained with Calcein AM for live cells and EthD-1 for dead cells 3 days after treatment and the number of EthD-1 positive organoids were manually counted from images taken from three wells of each condition at 4× magnification. All experiments were performed at least in triplicate. The mean values from each condition were compared by Student's paired or unpaired t-test using Graphpad Prism. *P<0.05, **P<0.01, ***P<0.001, ****P<0.0001.

### Results

### Pyrvinium Selectively Induces Cell Death of Mouse Dysplastic Organoids.

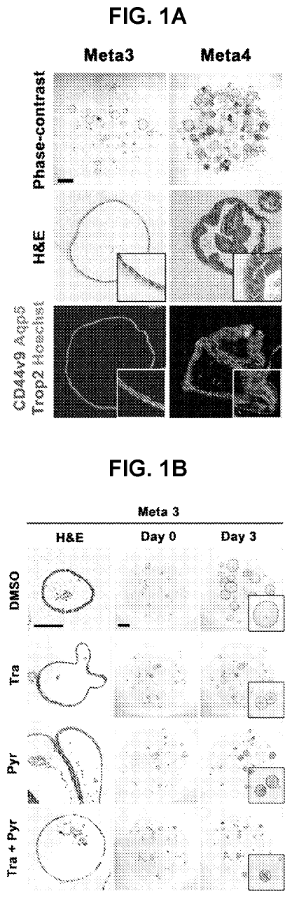

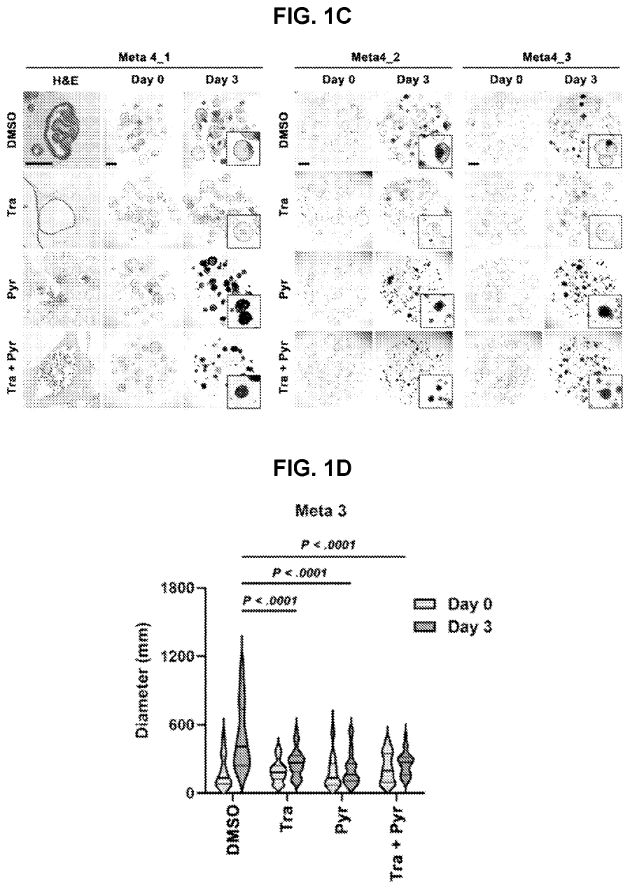

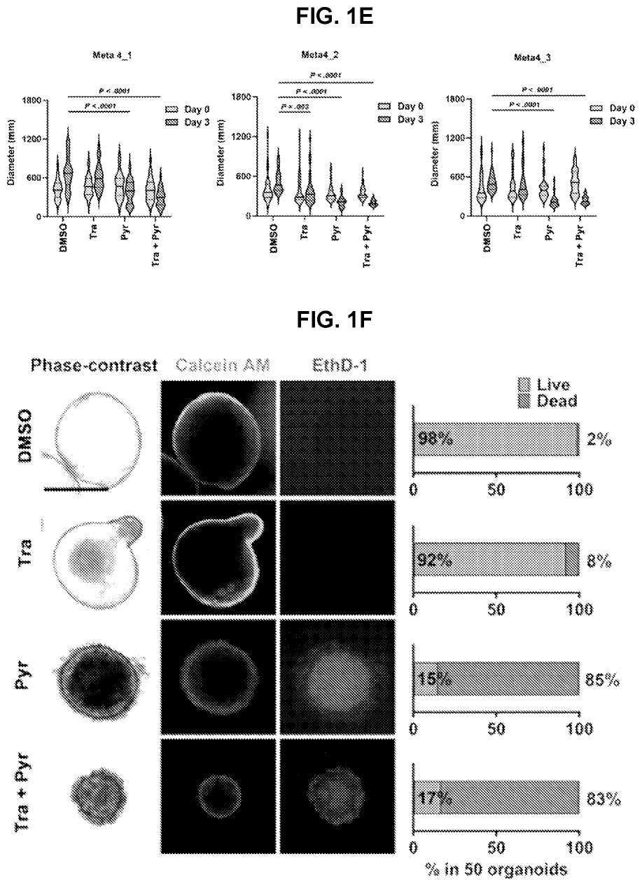

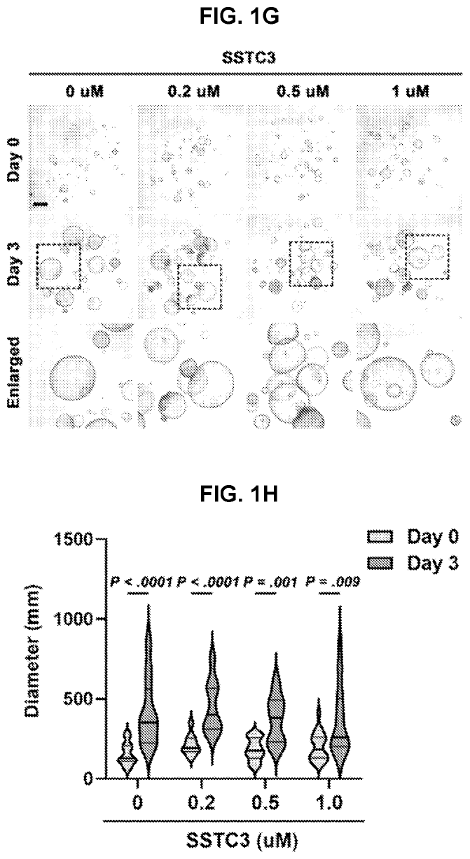

Previously, established metaplastic (Meta3) and dysplastic (Meta4) organoids were utilized to evaluate combined drug effects of pyrvinium, a putative Casein Kinase 1a (CK1a) activator, and trametinib, a MEK inhibitor, in Meta3 and Meta4 organoids. Meta3 expressed SPEM cell markers, Aquaporin 5 (AQP5) and CD44v9, while Meta4 organoids were specifically positive for a dysplastic cell marker, TROP2, as well as AQP5 (FIG. 1A). The Meta3 organoids displayed a significant reduction in size compared to control organoids treated with DMSO in response to trametinib, pyrvinium, and both (FIG. 1B and FIG. 1D). In contrast, the Meta4 organoids showed varying responses to trametinib and pyrvinium. Trametinib treatment only suppressed dysplastic organoid growth and induced a significant decrease in organoid diameter in one Meta4 line, Meta4_2 (FIG. 1C and FIG. 1E). Although other Meta 4 organoid lines showed no differences in size after the trametinib treatment, the organoid cell heights were thinner compared with control organoids, indicating lower cellularity as shown in H&E images (FIG. 1C). pyrvinium treatment not only led to a reduction in organoid size but also extensive cell death in all three Meta4 lines, as shown by numerous pyknotic cells in phase-contrast images (FIG. 1C) and confirmed by live/dead cell staining (FIG. 1F). Interestingly, co-treatment with trametinib and pyrvinium did not provide any additive or synergistic effects on Meta4 organoids (FIG. 1C and FIG. 1E). Taken together, these results suggest that pyrvinium can induce cell death in dysplastic organoids and inhibit growth of metaplastic organoids, but MEK inhibition can only lead to growth arrest in both metaplastic and dysplastic organoids.

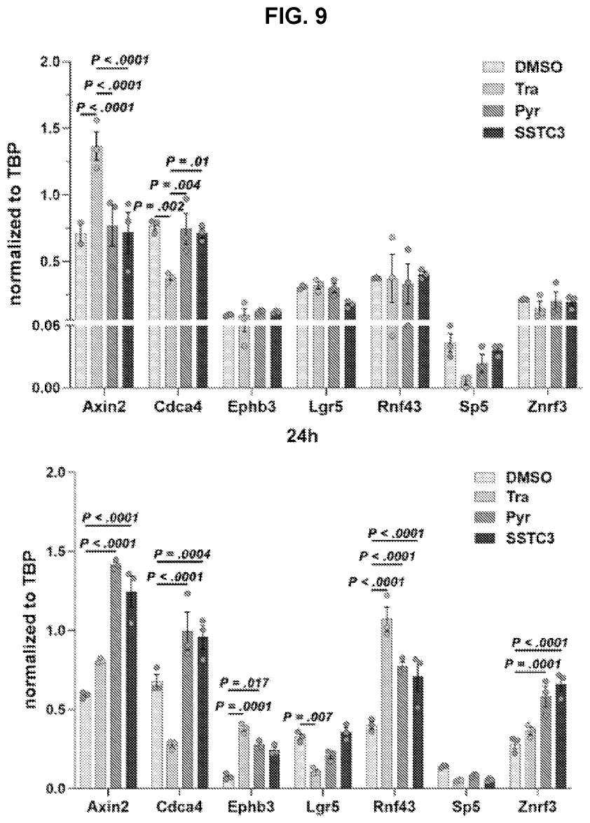

It was hypothesized that pyrvinium might have controlled the dysplastic cell survival and maintenance through activation of CK1a and functioned as a downstream of inhibitor of the Wnt pathway. To further evaluate whether the CK1a activation is influences the survival of dysplastic organoids, Meta4 was treated with SSTC3, a CK1α-specific activator. Surprisingly, SSTC3 did not cause any growth suppression or cell death in Meta4 organoids (FIG. 1G). Wnt-target genes were not downregulated after the pyrvinium treatment and the expression of several genes, such as Axin2, Cdca4, Rnf43, and Znrf3, were increased 24 hours after treatment (FIG. 9). Therefore, the results indicate that pyrvinium does not negatively impact the Wnt signaling pathway in dysplastic gastric cells.

### Pyrvinium Affects Both MAPK and STAT Pathways in Mouse Metaplastic and Dysplastic Organoids

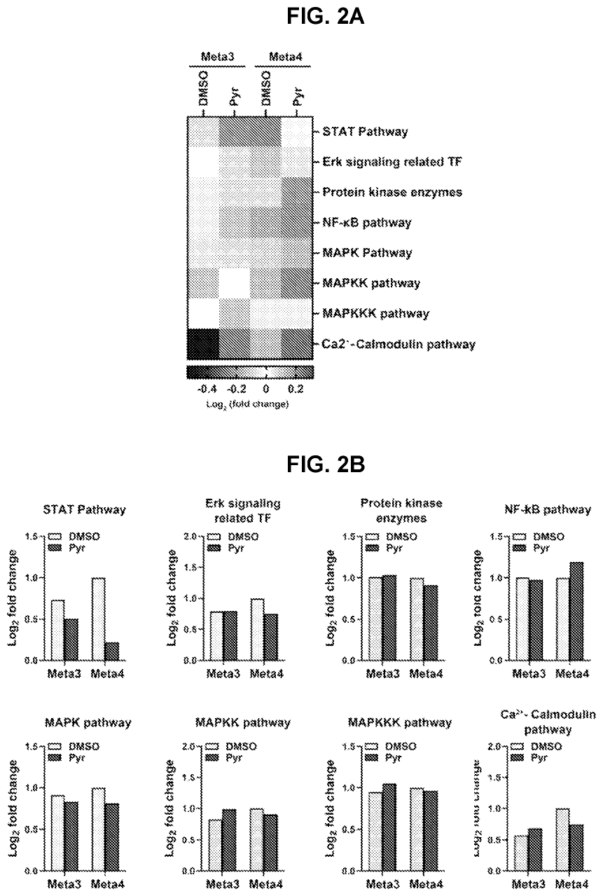

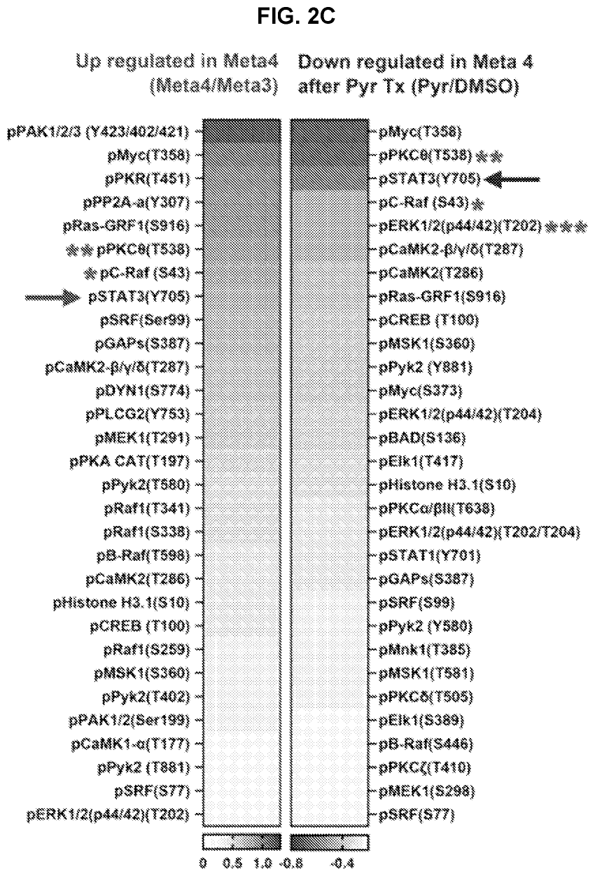

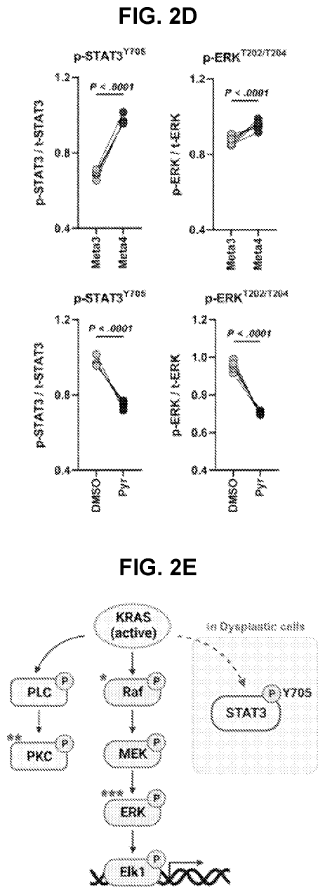

To identify what signaling pathways are controlled by pyrvinium in Meta4 organoids, a phospho-array analysis was conducted using Meta3 and Meta4 organoids treated with either DMSO vehicle or pyrvinium. Paired analysis between Meta3 and Meta4 organoids with or without pyrvinium treatment displayed differential profiles of protein phosphorylation, which includes STAT, ERK signaling related transcription factor, protein kinase enzymes, NF-κB, and MAPK pathways (FIG. 2A). In particular, the most prominent effect of pyrvinium treatment was dephosphorylation of proteins related to the STAT pathway (FIG. 2B). To identify potential proteins responsible for pyrvinium-induced cell death in Meta4 organoids, it was postulated that phosphoprotein candidates upregulated in Meta4 organoids would exhibit a significant reduction in phosphorylation upon pyrvinium treatment. The top 10 protein candidates were selected, including p-MYC^{T358}, p-PKCtheta^{T538}, p-C-Raf^{S43} p-STAT3^{Y705}, p-Pyk2^{T580}, p-Histone H3.1^{S10}, p-CREB^{T100}, p-Pyk2^{T881}, p-SRF^{S77}, and p-ERK1/2(p44/42)^{T202}(FIG. 2C-2D). As the organoids were derived from Mist1-Kras mice, it is not surprising that many proteins on the list are directly related to MAPK and PKC signaling pathways, with the exception of STAT3 (FIG. 2E). Therefore, these results suggest that STAT3 signaling is upregulated in dysplastic cells and might be an important pathway for dysplastic cell survival.

### Pyrvinium Induces Cell Death in Dysplastic Organoids Through Dual-Blockade of ERK and STAT3 Signaling Pathways

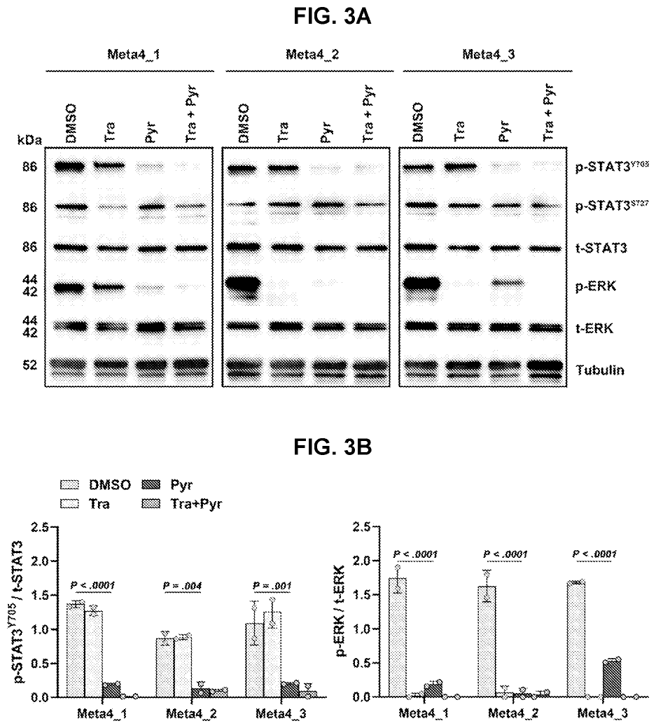

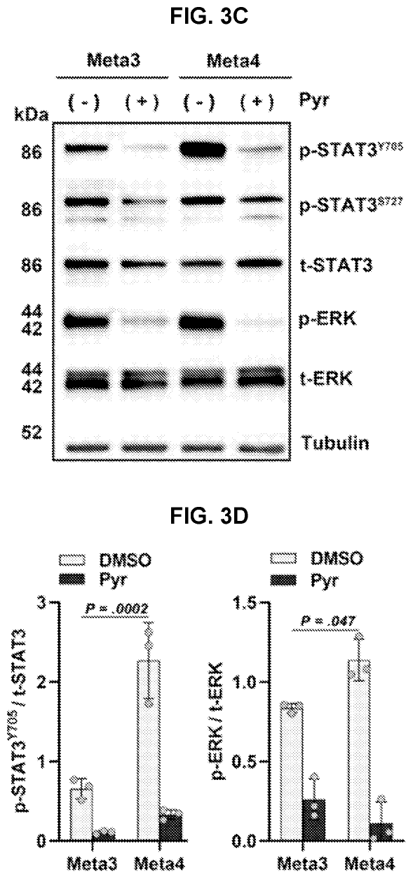

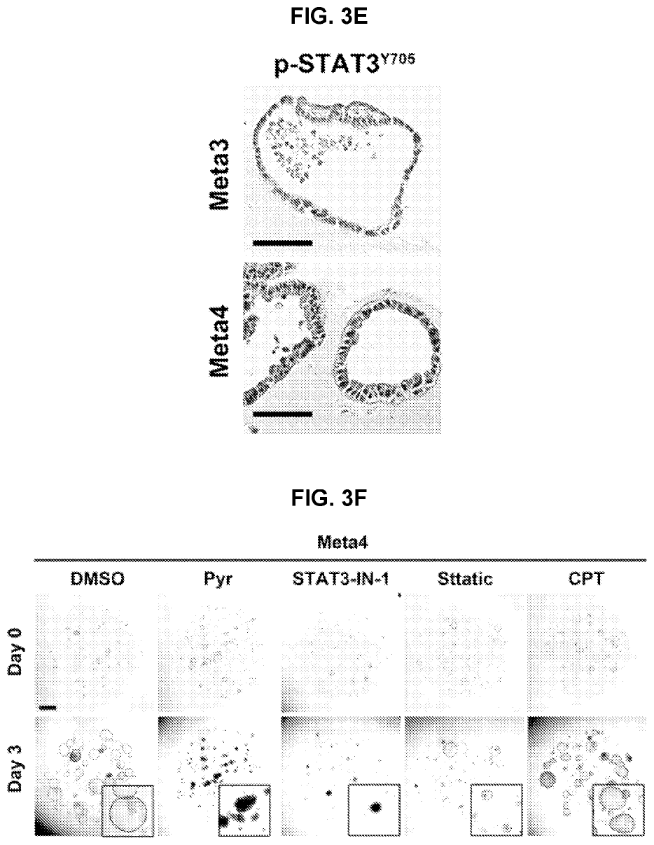

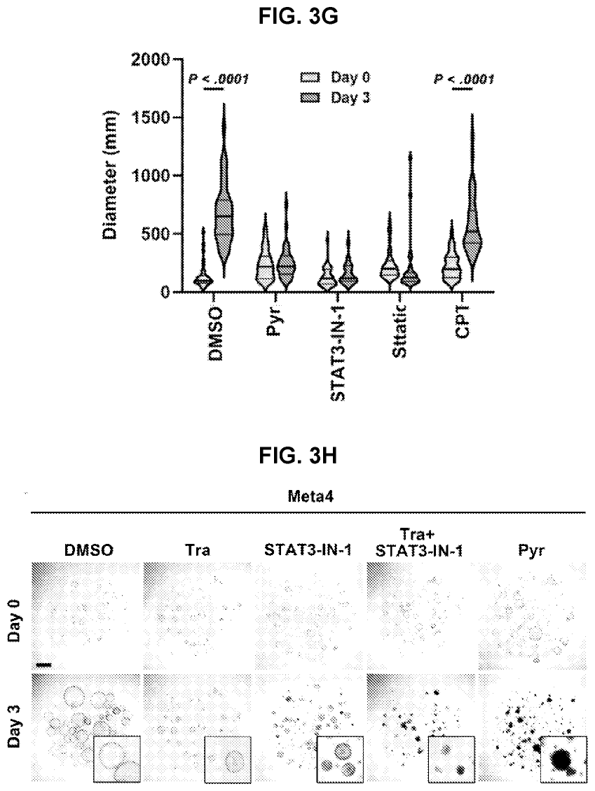

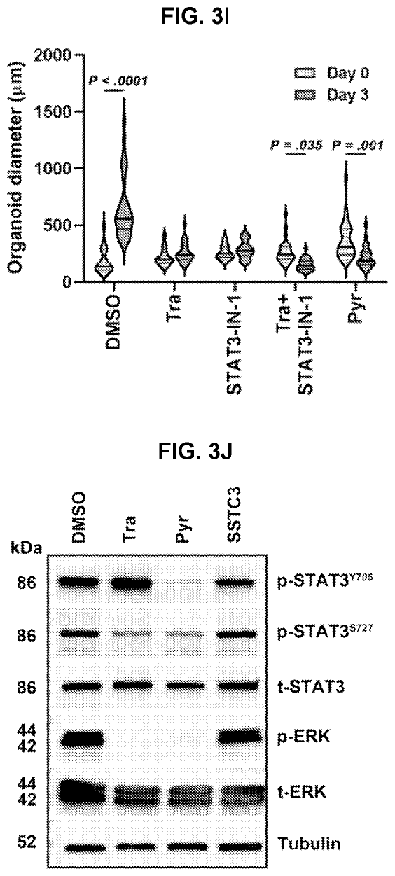

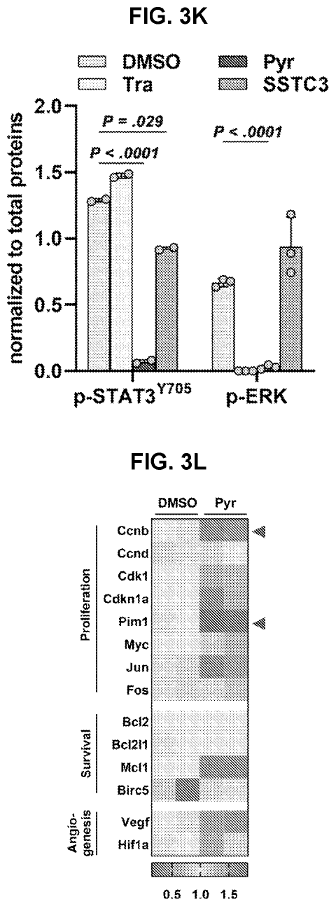

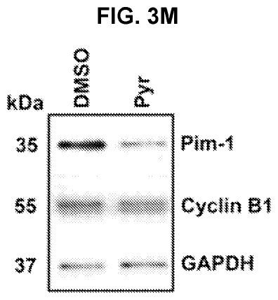

Dysregulated STAT3 signaling pathway has been strongly implicated in tumorigenesis through effects on cell growth, angiogenesis, immune system evasion, and prevention of apoptosis. It was therefore investigated how pyrvinium regulates the STAT3 activity in dysplastic organoids. STAT3 activation is typically induced by phosphorylation on either a tyrosine residue (pSTAT3^{Y705}) or a serine residue (p-STAT3^{S727}). The phospho-array data revealed a loss of STAT3^{Y705} phosphorylation after pyrvinium treatment. Western blot analysis confirmed a significant reduction in the phosphorylation of STAT3^{Y705}, but not STAT3^{S727}, in Meta4 organoids (FIG. 3A-3B). Of note, a significant decrease in p-ERK levels was observed after pyrvinium treatment, consistent with the results from the phospho-array analysis and trametinib also led to a significant reduction in p-ERK levels, as expected (FIG. 3A-3B). These findings suggest that dual suppression of STAT3^{Y705} and ERK phosphorylation is necessary to induce cell death in dysplastic organoids. Meta3 organoids also displayed reduced phosphorylation of both STAT3^{Y705} and ERK upon pyrvinium treatment (FIG. 3C). However, the basal phosphorylation level of STAT3^{Y705} in Meta3 organoids was significantly lower than that of Meta4 organoids (FIGS. 3C-3E) and the organoids did not exhibit any cell death, as shown previously.

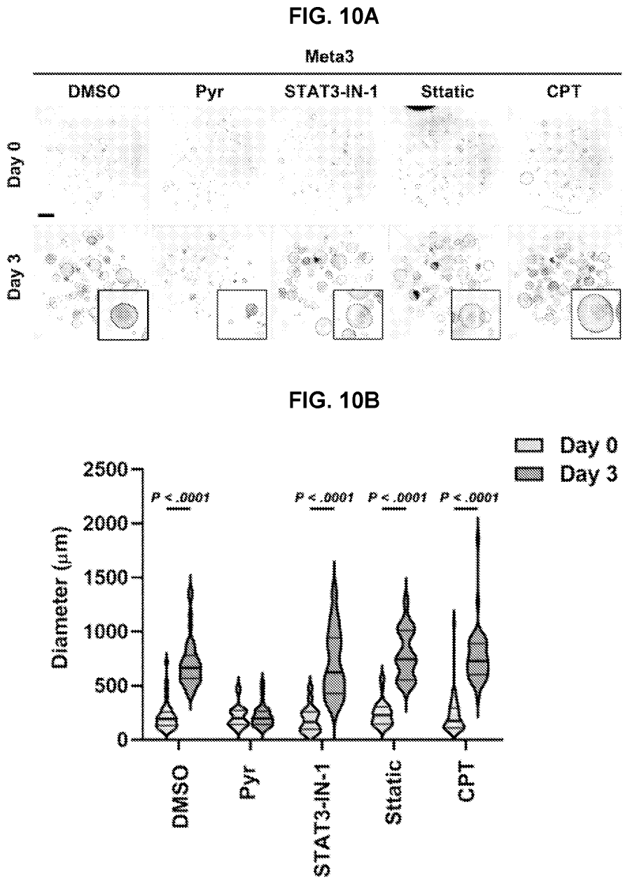

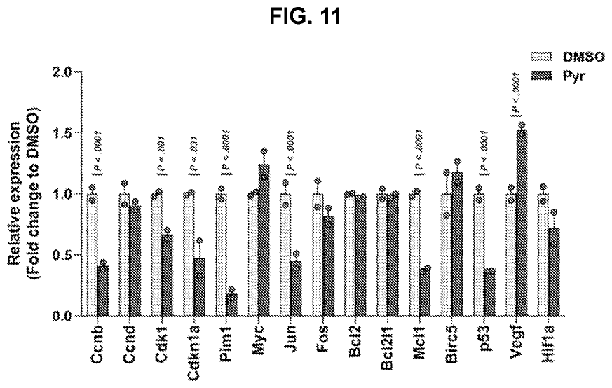

Additionally, the Meta3 and Meta4 organoids were treated with three different STAT3-specific inhibitors, including STAT3-IN-1, Sttatic, and Cryptotanshinone (CPT). All STAT3-specific inhibitors showed no influence on the survival and growth of Meta3 organoids (FIG. 10A-10B), indicating growth arrest induced by pyrvinium is mediated only by suppression of MEK/ERK signaling in metaplastic cells. Two STAT3-specific inhibitors, STAT3-IN-1 and Sttatic, induced growth arrest in Meta4 organoids, but did not lead to cell death (FIG. 3F-3G). In contrast, co-treatment with trametinib and STAT3-IN-1 resulted in significant cell death in Meta4 organoids, comparable to the effect of pyrvinium (FIG. 3H-3I). It was confirmed that SSTC3 treatment did not reduce p-STAT3^{Y705} or p-ERK expression confirming that pyrvinium action is not through CK1a activation (FIG. 3J-3K). Additionally, changes in STAT3-related genes were examined in response to pyrvinium treatment. The expression of several STAT3-target genes, including Cyclin B1 and Pim-1, was downregulated after pyrvinium treatment (FIG. 3L and FIG. 11), which was also confirmed at the protein level (FIG. 3M). Pim-1 is overexpressed in various human cancers and protects against cell apoptosis. Thus, the downregulation of Pim-1 might be associated with the induction of cell death in dysplastic cells. Consequently, the results indicate that a dual inhibition of both ERK and STAT3 signaling pathways is critical for dysplastic cell survival.

Pyrvinium Targets CD133⁺CD166⁺ Dysplastic Stem Cells

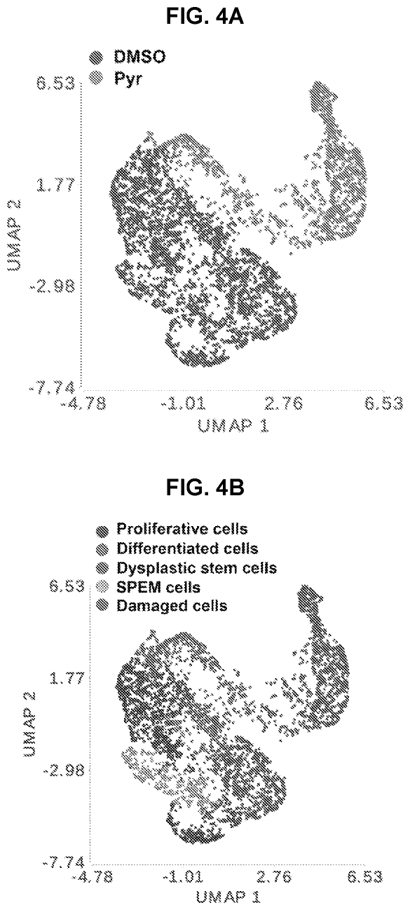

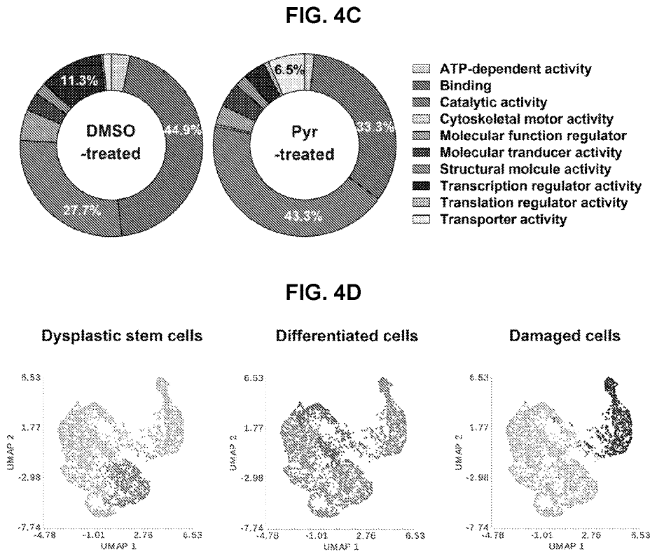

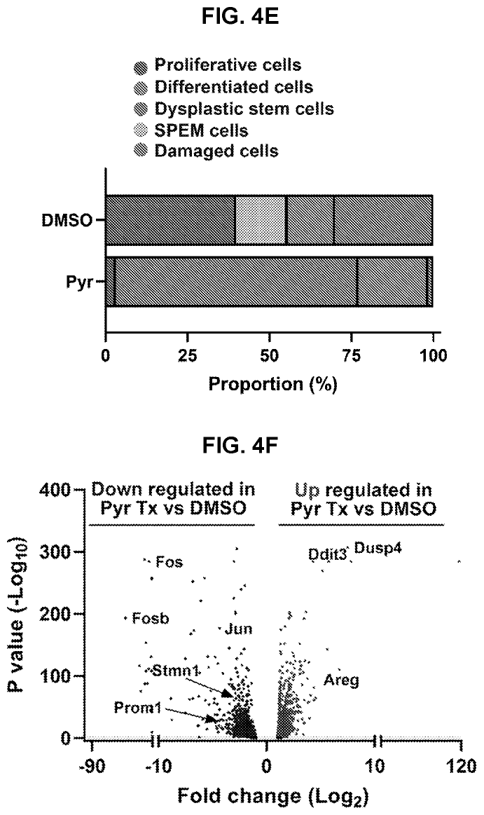

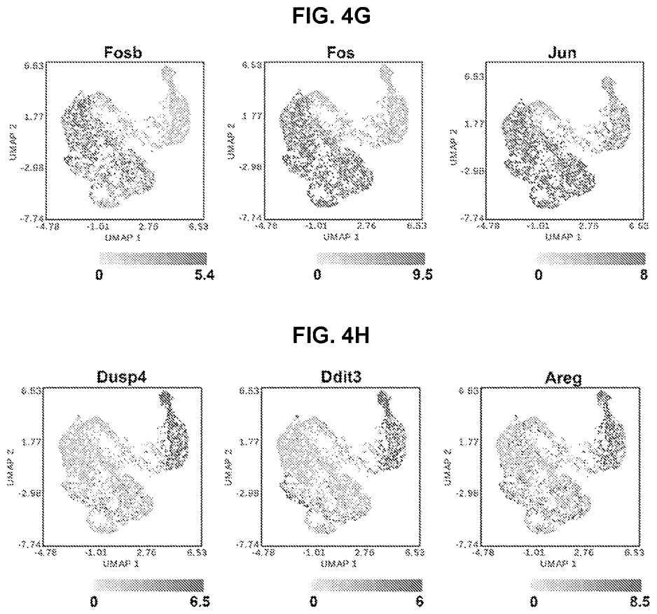

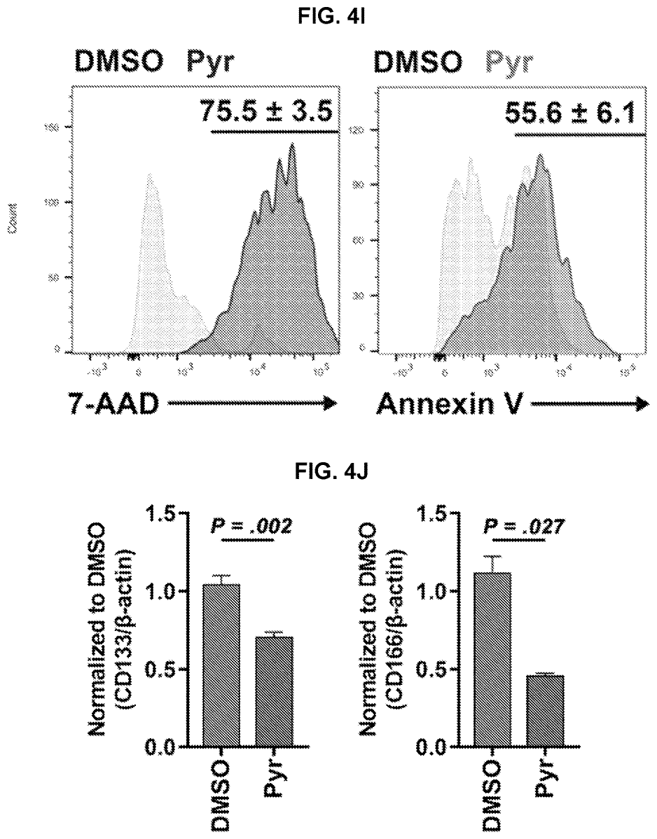

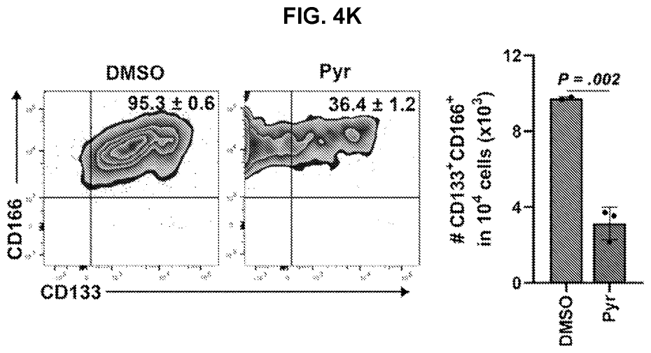

To comprehensively evaluate transcriptomic alterations and identify subpopulations of dysplastic organoids that are affected by pyrvinium treatment, single-cell RNA-sequencing was performed using cells from Meta4 organoids treated with either DMSO or pyrvinium for 1 day before organoids exhibited morphological disruption. Uniform Manifold Approximation and Projection (UMAP) demonstrated an almost complete separation between DMSO-treated and pyrvinium-treated cells, indicating dramatic alterations in transcription elicited by pyrvinium (FIG. 4A). Five subpopulations were determined by unsupervised clustering combined with lineage specific markers including Stmn1 and Pcna for proliferative cells, TFF3 for differentiated intestinal-type cells, CD133 and CD166 for DSCs, Ma/2 and Lgals3 for SPEM cells, and Ddit3 and Areg for damaged cells (FIG. 4B). Gene ontology analysis combined with PANTHER classification demonstrated distinct gene expression profiles among the groups (FIG. 4C). pyrvinium treatment led to increased catalytic and transporter activities, whereas decreased binding and transcription regulator activities. While many subpopulations such as proliferative cells, SPEM cells, and DSCs were found in DMSO-treated cells, over 73.9% of cells were damaged and mostly differentiated cells among pyrvinium-treated cells (FIG. 4D-4E). Volcano plot and UMAP analysis indicated that several STAT3-target genes observed in DMSO-treated cells, such as Fos, Fosb, and Jun, were downregulated after pyrvinium treatment (FIG. 4F-4G), while damage-related genes such as Dusp4, Ddit3 and Areg were upregulated in pyrvinium-treated cells (FIG. 4F-4H). Furthermore, an increase in both 7-AAD and Annexin V expression was observed in the pyrvinium-treated cells, confirming increased activities in necrosis and apoptosis in response to pyrvinium (FIG. 4I). It is important to note that the DSC subpopulation was not observed after pyrvinium treatment. qRT-PCR analysis validated decreases in CD133 and CD166 gene expression (FIG. 4J) and FACS analysis also demonstrated a significant decrease in CD133⁺CD166⁺ DSCs (FIG. 4K). These findings indicate that pyrvinium specifically targets DSCs as well as proliferating cells, leading to cell death in dysplastic organoids.

### Pyrvinium Treatment Inhibits Metaplasia Progression to Dysplasia in the Mouse Stomach

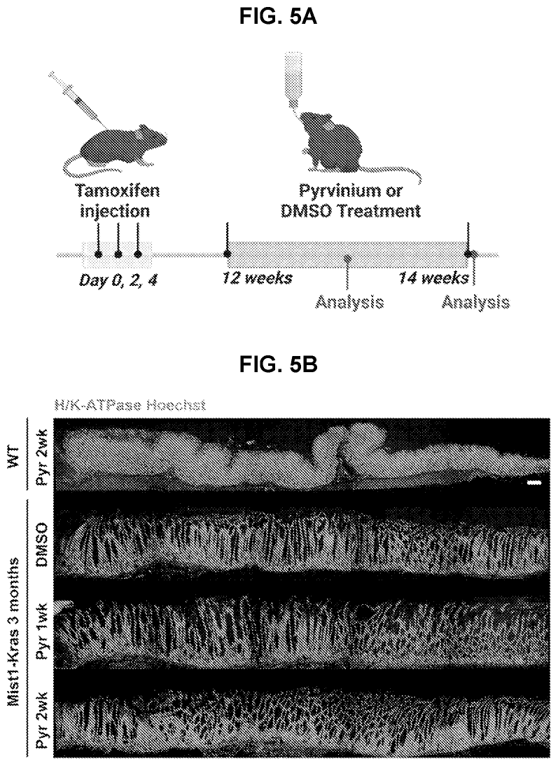

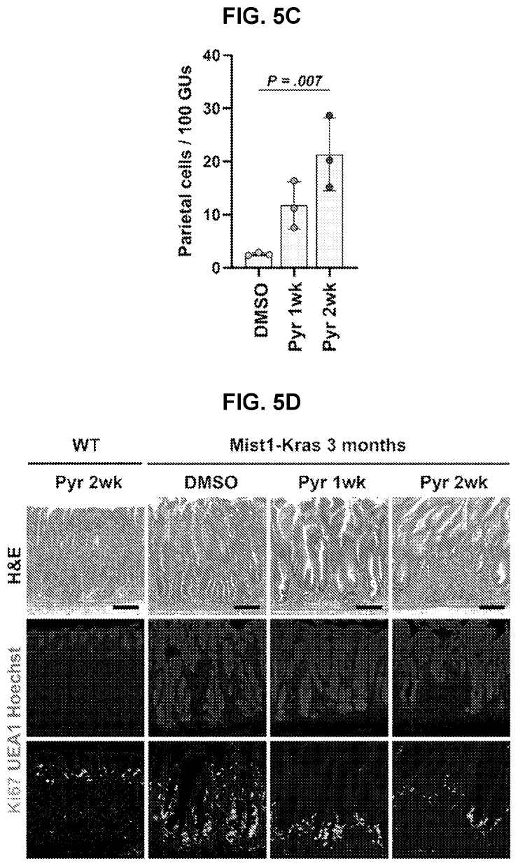

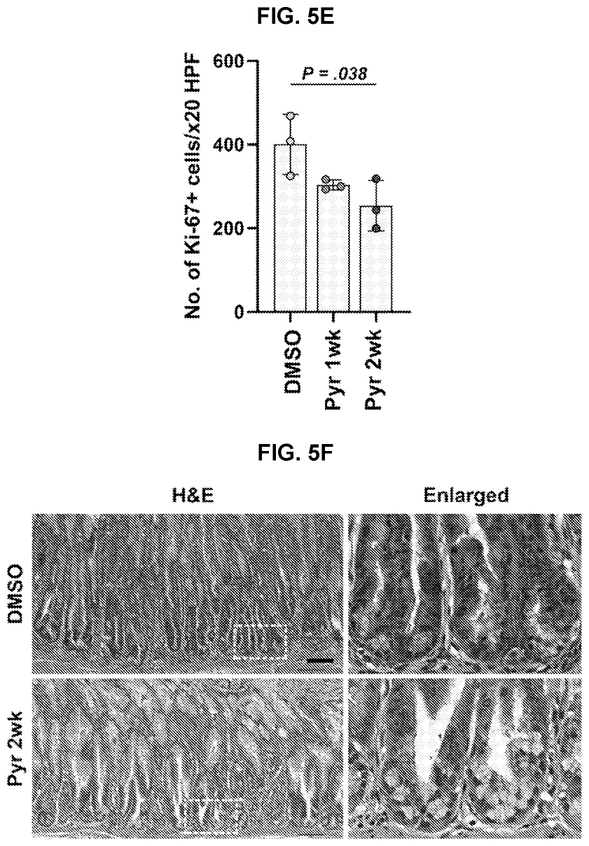

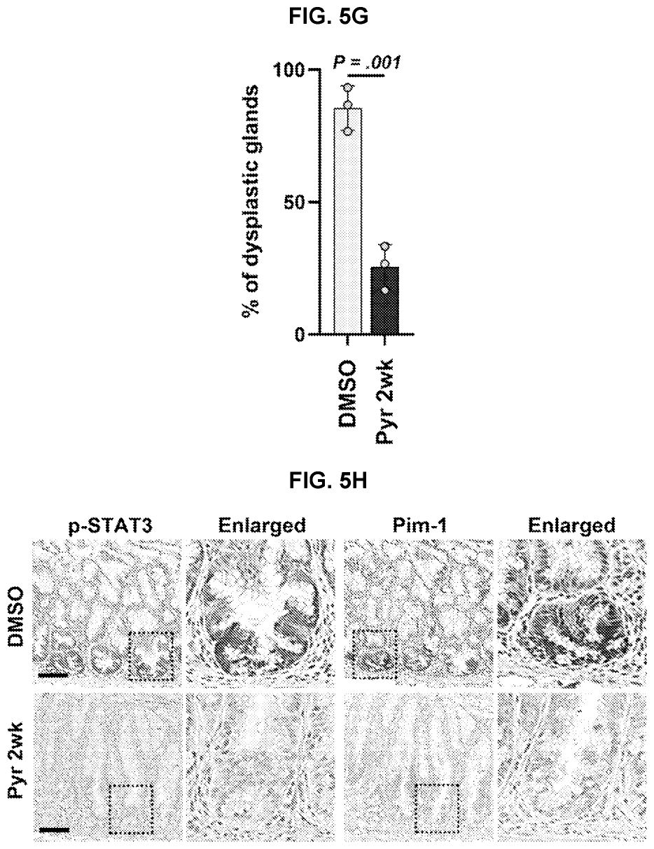

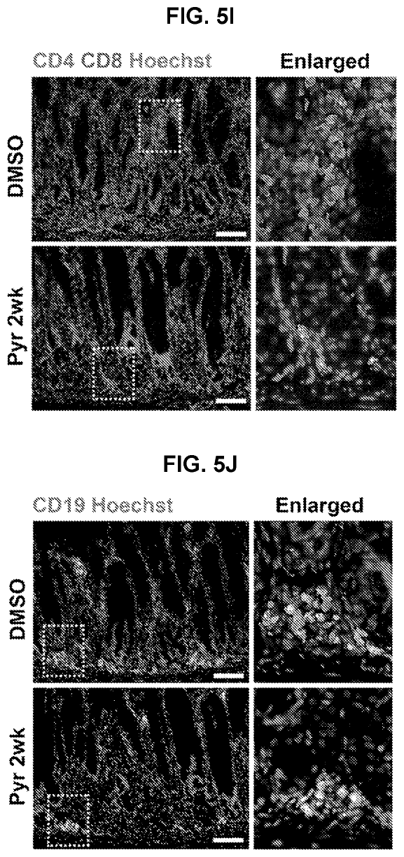

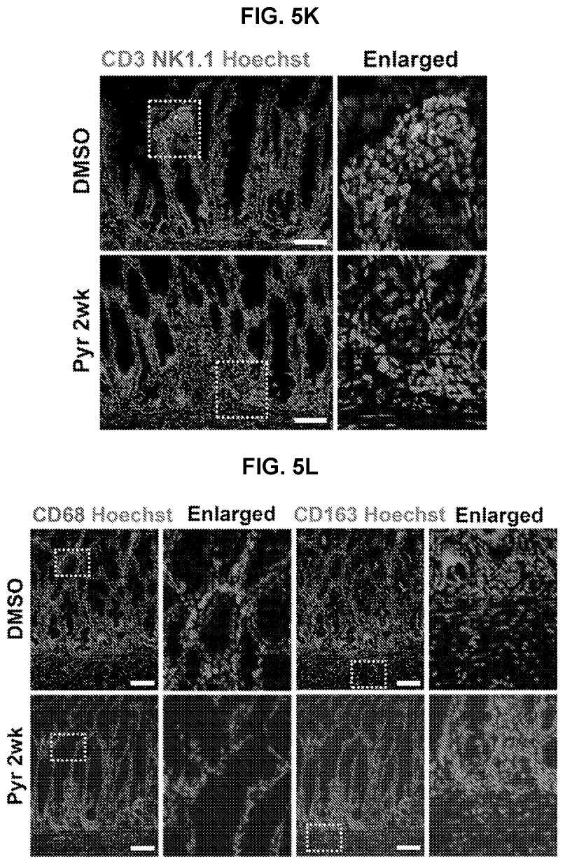

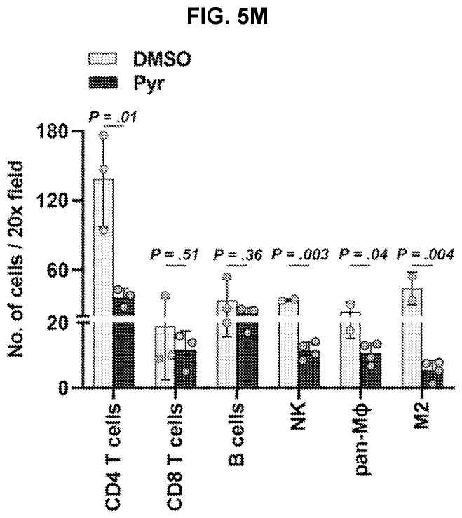

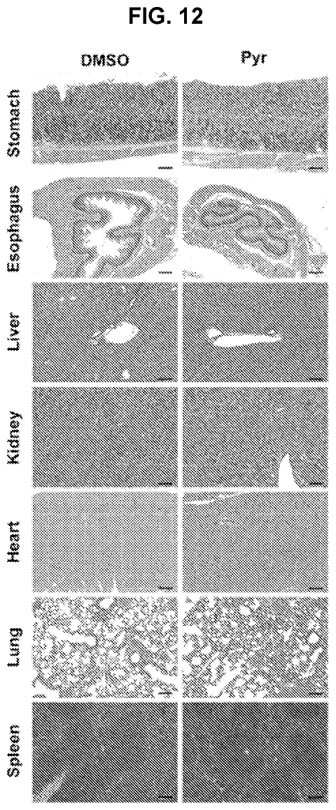

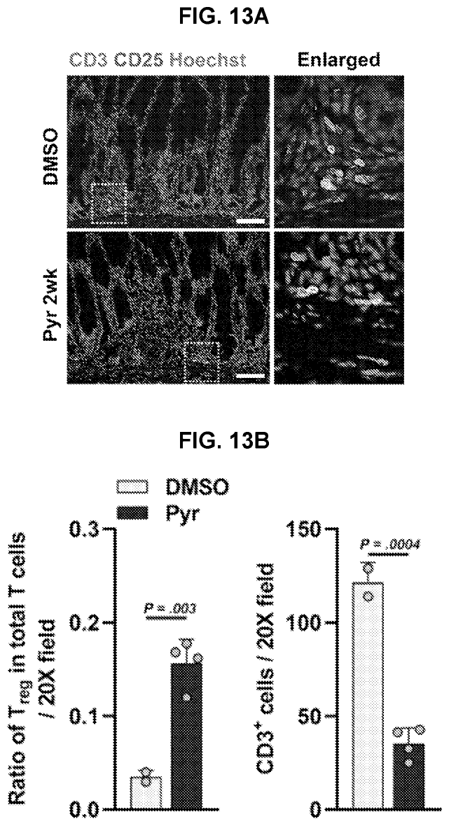

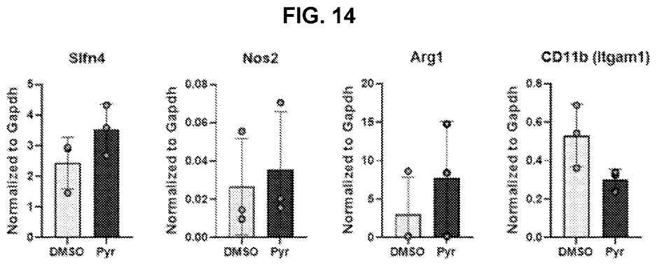

Next, it was investigated whether the STAT3 signaling pathway is upregulated during metaplasia progression to dysplasia in vivo. At 3 months after tamoxifen injection in Mist1-Kras mice, stomachs predominantly exhibited metaplastic glands, with an appearance of dysplastic glands becoming increasingly prevalent by four months. The mice were therefore treated at 3 months after tamoxifen injection and evaluated whether pyrvinium could ameliorate metaplasia progression to dysplasia (FIG. 5A). One or two weeks after either DMSO or pyrvinium treatment, gastric mucosa in each group of mice was examined. Acid-secreting parietal cells gradually repopulated the corpus mucosa in mice treated with pyrvinium (FIG. 5B-5C). Conversely, the number of hyperplastic foveolar glands with UEA1 and Ki-67 positive proliferating cells significantly decreased (FIG. 5D-5E), along with a significant decrease in the number of dysplastic glands (FIG. 5F-5G). Furthermore, pyrvinium treatment resulted in downregulation of p-STAT3 and Pim-1 (FIG. 5H), as observed in dysplastic organoids (FIG. 3L-3M). We additionally conducted a comprehensive examination of major organs in wild-type mice treated with either DMSO or pyrvinium and confirmed that pyrvinium did not induce any noticeable histopathological abnormalities (FIG. 12). Alterations in the immune microenvironment after pyrvinium treatment were further explored. Mice treated with DMSO, exhibited various inflammatory cell infiltration, such as lymphocytes and macrophages, in the lamina propria. pyrvinium treatment induced a dramatic decrease in the CD4⁺ T cell population (FIG. 5I), but it did not show a significant difference in CD8⁺ T or CD19⁺ B cells (FIG. 5I and FIG. 5J). M2-macrophages have been shown to promote progression of SPEM and IM, and both NK cells and CD163-positive M2-macrophages significantly decreased in pyrvinium-treated mice (FIGS. 5K-5M). While the total number of CD3⁺ T cells decreased in pyrvinium-treated mice, the relative ratio of CD3⁺CD25⁺ regulatory T cell number significantly increased (FIGS. 13A-13B) and the expression of myeloid-derived suppressor cell (MDSC)-related markers tended to increase, although the changes were not significant (FIG. 14). These results suggest that pyrvinium effectively inhibits metaplasia progression to dysplasia and promotes the regeneration of normal gastric mucosa in vivo by contributing an anti-inflammatory microenvironment.

Pyrvinium Inhibits Growth and Survival of Human Precancerous Cells with Dysplastic Features

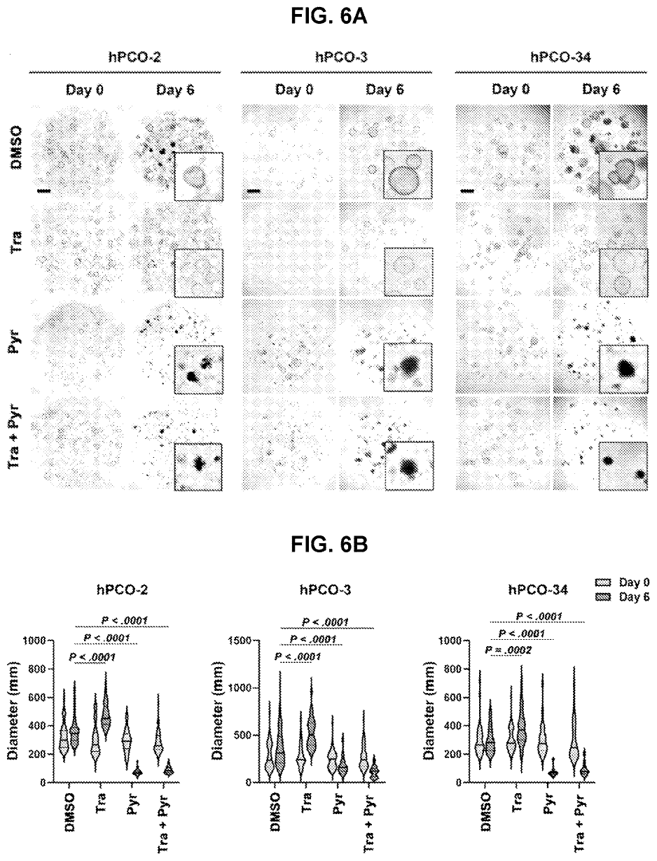

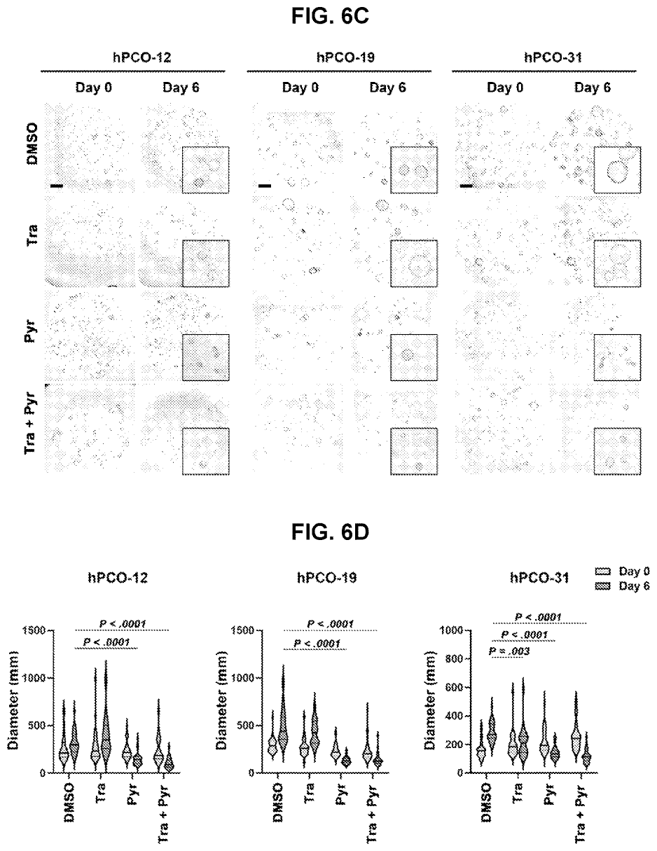

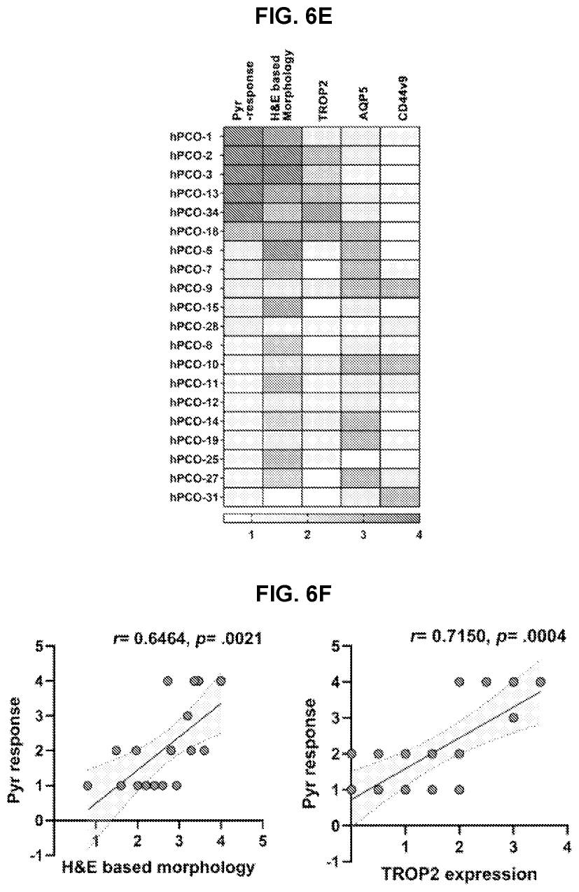

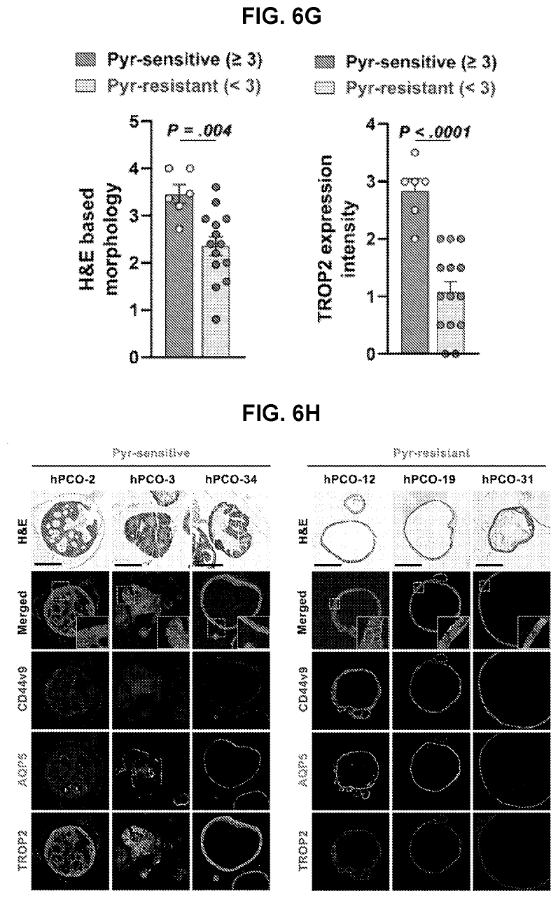

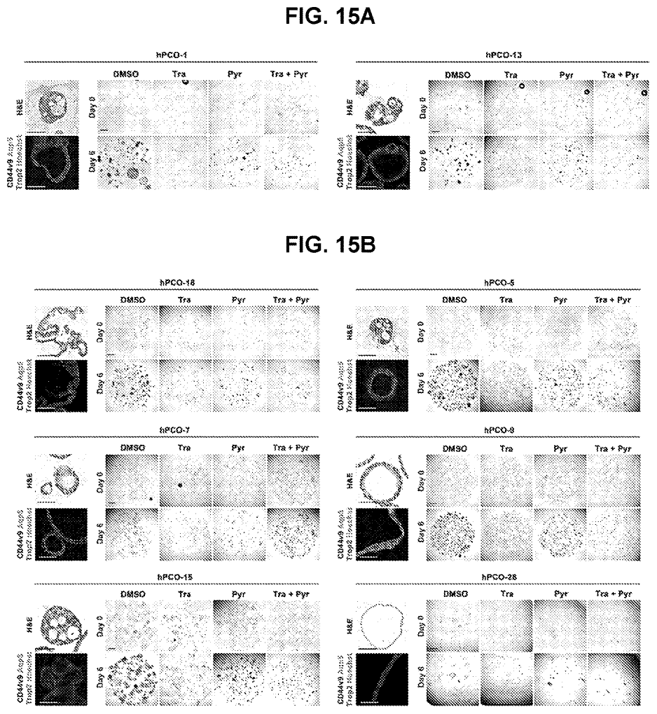

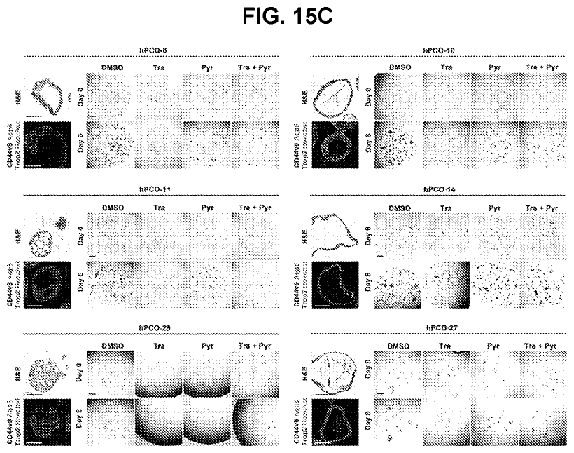

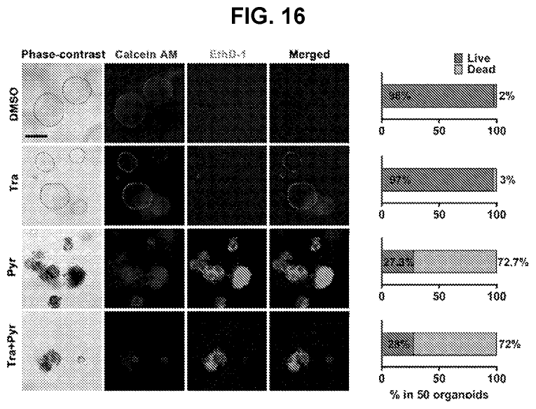

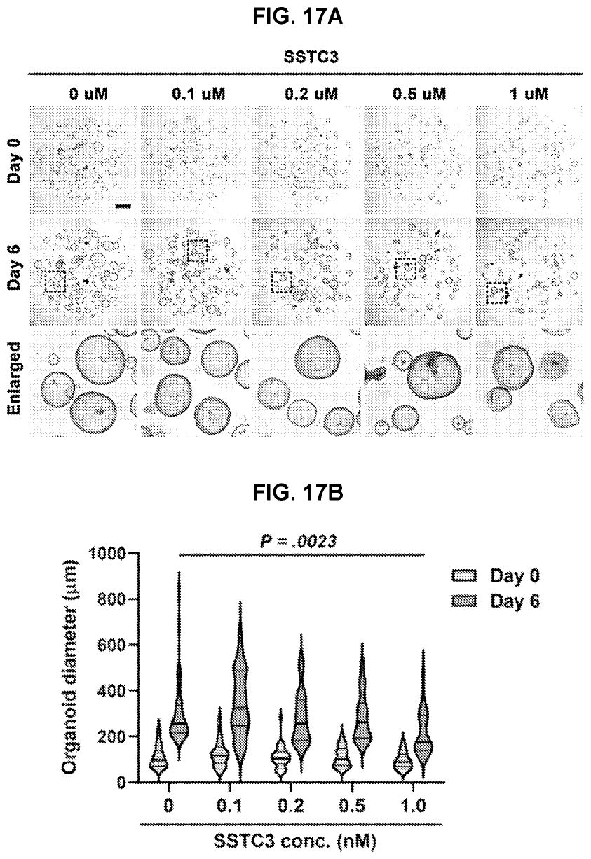

It is known that non-tumor-bearing mucosa surrounding gastric cancer frequently harbors metaplastic or dysplastic glands with varying degrees of genetic or epigenetic abnormalities, a phenomenon known as “field cancerization.” To investigate the inhibitory effects of pyrvinium on the growth and survival of these human pre-cancerous lesions, twenty human pre-cancerous organoid (hPCO) lines were established from gastric mucosa adjacent to gastric cancer obtained from surgical specimens. The hPCO lines were characterized based on morphology and expression profiles of AQP5 and CD44v9 as metaplastic cell markers and TROP2 as a dysplastic cell marker (FIG. 6E). Following treatment with trametinib, pyrvinium, and their combination for 6 days, the diameters of organoids were measured, and organoid death was examined, categorized by scores, ranging from 1 to 4 (where 0 represents no death, and 1, 2, 3, and 4 correspond to 0-10%, 10-50%, 50-90%, and >90% deceased organoids, respectively). Based on these results, the hPCO lines were classified into three categories: sensitive (score 4), moderate (score 2 or 3), and resistant (score 0 or 1) groups (FIG. 6E). Five pyrvinium-sensitive hPCO lines displayed significant organoid death, while nine pyrvinium-resistant hPCO lines remained mostly alive with no or a few dead cells and six pyrvinium-moderate lines exhibited mild to moderate extent of cell death (FIG. 6 and FIG. 15A-15C). Three representative images from pyrvinium-sensitive hPCO lines (hPCO-2, hPCO-3, and hPCO-34) are shown in FIG. 6A, and the organoid death was confirmed by live/dead cell staining using the hPCO-34 line (FIG. 16). pyrvinium induced organoid death in pyrvinium-sensitive lines, but co-treatment with trametinib did not show any additional effects, as observed in the mouse dysplastic organoids (FIG. 1C). Interestingly, pyrvinium-sensitive lines did not respond to trametinib and continuously grew for 6 days, which is in contrast to the growth inhibition observed in mouse organoids after trametinib treatment (FIG. 6A-6B). In addition, pyrvinium-resistant hPCO lines (hPCO-12, hPCO-19, and hPCO31) did not die, but still exhibited growth arrest after the treatment with pyrvinium and/or trametinib (FIG. 6C-6D). Additionally, it was evaluated whether the drug responses in different human organoids are associated with their histological and molecular characteristics (FIG. 6E). Remarkably, it was observed that pyrvinium-sensitive hPCO lines exhibited positive correlations with dysplastic morphology and high levels of TROP2 expression, whereas they demonstrated negative correlations with AQP5 and CD44v9 expression (FIG. 6F-6H). Collectively, the findings suggest that the pyrvinium effects are significantly associated with dysplastic features of hPCO lines.

### Pyrvinium Blocks Both ERK and STAT3 Signaling Pathways in Human Dysplastic Cells

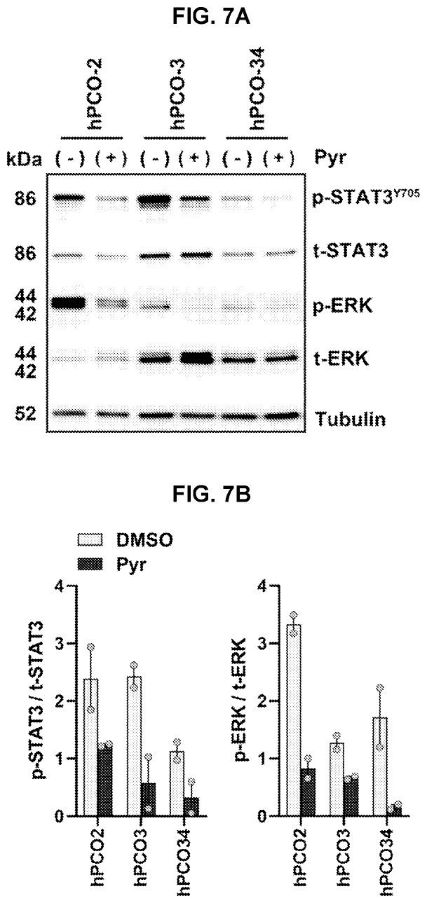

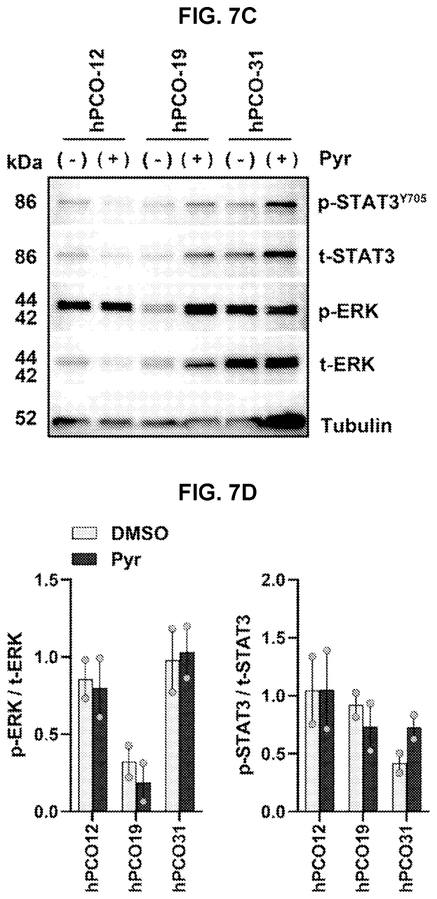

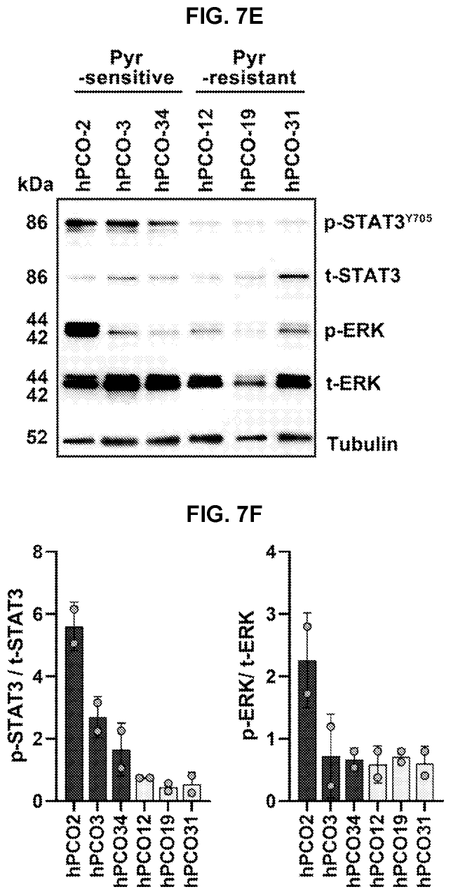

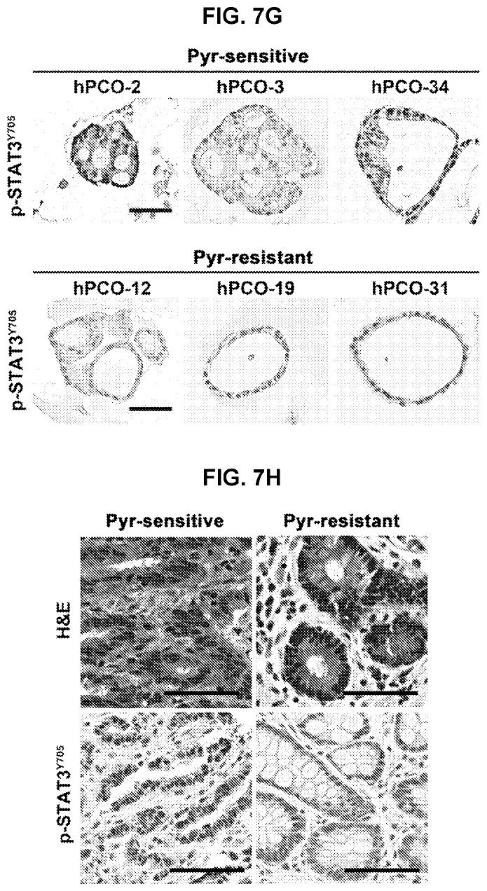

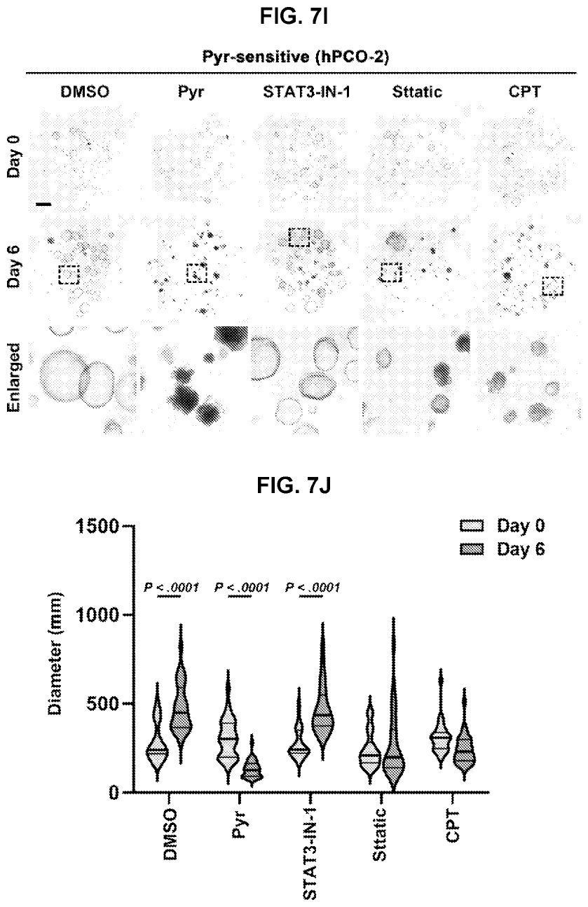

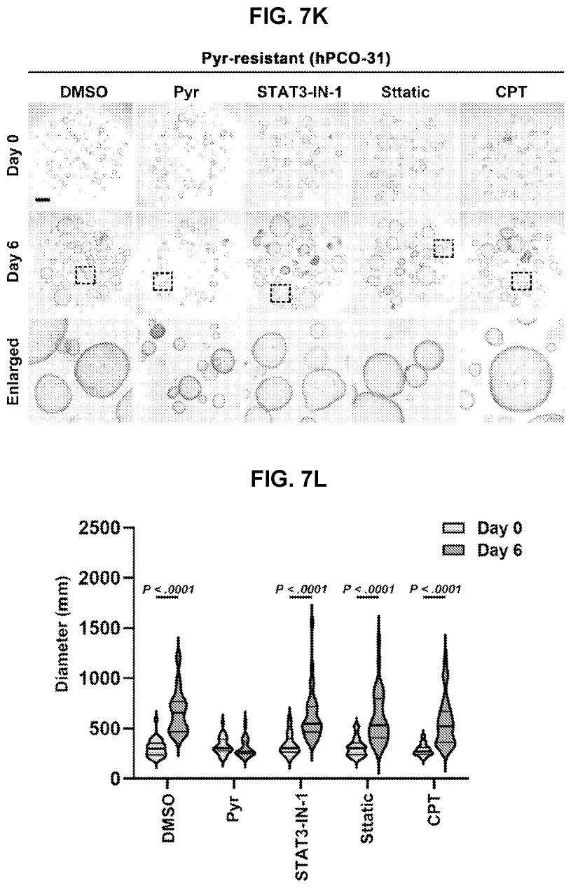

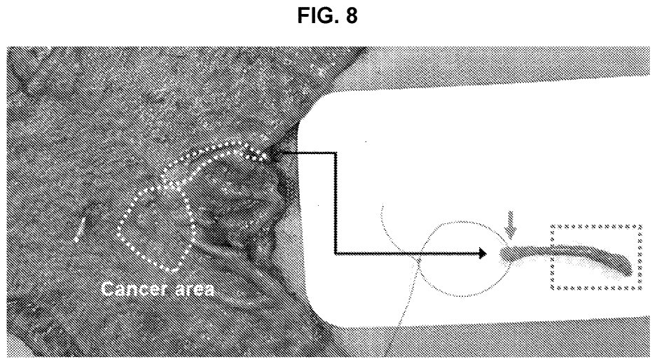

The relationship between the pyrvinium responsiveness and the STAT3 and ERK signaling pathway activation in hPCO lines was further examined. Changes in phosphorylation levels of STAT3^{Y705} and ERK were examined in three pyrvinium-sensitive and pyrvinium-resistant hPCO lines after pyrvinium treatment. Pyrvinium treatment decreased both p-STAT3^{Y705} and p-ERK levels in all three pyrvinium-sensitive hPCO lines (FIG. 7A-7B), whereas no significant differences were observed in the pyrvinium-resistant group (FIG. 7C-7D). Like the findings in mouse organoids, upregulation in STAT3^{Y705} phosphorylation was also observed in the pyrvinium-sensitive hPCO lines compared to the pyrvinium-resistant group (FIG. 7E-7G), as well as in the gastric tissues used for the hPCO establishment (FIG. 7H). Additionally, hPCO lines were treated with three STAT3-specific inhibitors and assessed their effects on the growth and survival in hPCO lines. Although two STAT3 inhibitors, Sttatic or CPT, but not STAT3-IN-1, arrested organoid growth in pyrvinium-sensitive hPCO-2 line, the organoids survived until 6 days after the treatment (FIG. 7I-7J). In contrast, pyrvinium-resistant hPCO-31 did not respond to all STAT3 inhibitors (FIG. 7K-7L). Thus, these results suggest that pyrvinium can effectively suppress the growth and survival of hPCOs by simultaneously inhibiting both STAT3 and ERK signaling pathways.

### CONCLUSION

Long-lasting tissue injury can lead to multifocal occurrences of precancerous lesions, which accumulate genetic alterations over time and eventually develop multiple types of cancers in the stomach. Both severe metaplastic and dysplastic lesions are believed to be irreversible (point of no return). Therefore, it is imperative to develop chemo-preventive measures that directly target these precancerous lesions. However, few investigations have been performed in this area, due to the inherent difficulties in studying gastric precancerous cell expansion and progression in human. In this study, of mouse and human precancerous organoids were leveraged to define alterations in signaling pathways between metaplastic and dysplastic stages. It was observed that the STAT3 signaling pathway is dysregulated in the dysplastic stage and this caused different drug responses to pyrvinium. It is important to note that pyrvinium has been known as a Wnt pathway inhibitor by activating CK1a and regulate colon cancer cell survival. Also, several other signaling pathways, such as PI3K, Hedgehog, and Hippo can also be regulated by pyrvinium in various cancers. In the present study, they report that pyrvinium can also control the level of MEK/ERK phosphorylation and a dual blockade of both STAT3 and MEK/ERK signaling pathways, but not CK1a activation, leads to extensive cell death in dysplastic organoids. Therefore, appropriate usage of pyrvinium should be carefully considered based on its targets and functions in different organs and diseases.

Numerous data have demonstrated therapeutic effects of MEK inhibitors in various cancers. The previous and present studies display suppressive effects of MEK inhibitor, trametinib, on the growth in mouse dysplastic cells. However, most hPCO lines did not respond to trametinib and hPCO lines with dysplastic features even grew larger in size compared with vehicle-treated organoids. Human precancer organoid lines were established from heterogeneous tissues from gastric cancer patients and showed relatively lower phosphorylation levels of ERK. These findings indicate that MEK inhibition alone may not be enough to control human precancer progression. Previously, dysplastic stem cells, Trop2⁺CD133⁺CD166⁺ cells, were identified as being first present in the dysplastic stage in both mouse and human. Single cell-RNAseq data analysis revealed that pyrvinium specifically targets the CD133⁺/CD166⁺ stem cell population in dysplastic organoids. Furthermore, pyrvinium regressed in vivo metaplastic progression in Mist1-Kras stomachs. Metaplasia did not progress to dysplasia by targeting dysplastic stem cells, but normal gastric cells, especially parietal cells, were observed 2 weeks after pyrvinium treatment. Therefore, the drug response to pyrvinium in human precancerous lesions does not only depend on the phosphorylation level of both ERK and STAT3, but also the presence of dysplastic stem cells. Also, this study provides compelling evidence for pyrvinium as a potential therapeutic candidate that targets a stem cell population and can prevent gastric precancer progression to gastric cancer development.

In addition, strong activation of STAT3 signaling pathway upon MEK inhibition has been reported in KRAS mutant cancer cells and a dual inhibition of MEK and STAT3 has been demonstrated to be more effective to treat oncogene-addicted cells. Similarly, high phosphorylation levels of STAT3 were observed in both mouse and human dysplastic organoids, which strongly responded to pyrvinium. It has been suggested that pyrvinium's dramatic anticancer activity is mediated by its suppressive action on the STAT3 pathway in KRAS-mutant lung cancer and leukemia. However, in this study, the treatment of STAT3 inhibitor alone displayed various responses and much less effective in inducing cell death in dysplastic organoids, compared with pyrvinium treatment. All these findings suggest that dual blockade of MEK and STAT3 may be required to treat dysplasia in the stomach. Unlike dysplastic lesions, most metaplastic changes are benign, regenerative adaptations to severe injury. pyrvinium treatment did not induce apoptosis in metaplastic cells and only led to the growth arrest in both mouse metaplastic organoids and hPCO with metaplastic features, suggesting a lower likelihood of potential adverse effects on tissue regeneration. However, trametinib, a MEK inhibitor, and STAT3 inhibitors failed to limit the growth of metaplastic cells in hPCO lines, either. Since gastric metaplasia is considered as a precancerous lesion associated with dysplasia development, it would be crucial to reduce the risk of overgrowth of metaplasia without compromising its protective effect.

The development of direct pharmacological interventions for targeting precancerous lesions represents a promising strategy for cancer prevention. However, identifying specific signaling pathways that are crucial for high-risk precancerous lesions remains one of the most challenging issues in this field. This study concludes that MEK/ERK and STAT3 signaling pathways are differentially activated in gastric precancerous lesions. Also, pyrvinium exerted a dual blockade of MEK/ERK and STAT3, inducing significant cell death in STAT3 dysregulated dysplastic cells. Taken together, the repurposing of pyrvinium may present a meaningful therapeutic opportunity for patients who harbor gastric precancerous lesions.

1. A method for reprogramming a pre-cancerous mucosa to a non-cancerous state, the method comprising:
identifying a pre-cancerous mucosa comprising a metaplastic cell or a dysplastic cell; and contacting the metaplastic cell or the dysplastic cell with a compound of formula (I), or a salt thereof: 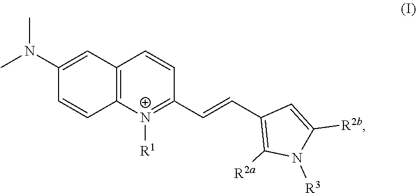
wherein:
R¹ is C₁₋₆alkyl;
R^{2a} and R^{2b} are each independently C₁₋₆alkyl; and
R³ is C₆₋₁₂aryl, wherein R³ is optionally substituted with 1-5 R^{X};
R^{X}, at each occurrence, is independently C₁₋₆alkyl, C₁₋₄haloalkyl, halogen, cyano, —N(R^{Xa})₂, —OR^{Xb}, —SR^{Xb}, —C(O)R^{Xb}, —CO₂R^{Xb}, —C(O)N(R^{Xa})₂, —SO₂R^{Xa}, -L²-Y², —O-L^{X}-Y^{X}, —S-L^{X}-Y^{X}, or —N(R^{Xa})-L^{X}-Y^{X};
L^{X}, at each occurrence, is independently C₁₋₆alkylene, C₂₋₆alkenylene, or C₂₋₆alkynylene;
Y^{X}, at each occurrence, is independently hydrogen, cyano, halogen, haloalkyl, —OH, —N(R^{Xa})₂, —OR^{Xb}, —SR^{Xb}, —C(O)R^{Xb}, —CO₂R^{Xb}, —C(O)N(R^{Xa})₂, or —SO₂R^{Xa};
R^{Xa}, at each occurrence, is independently hydrogen, C₁₋₄alkyl, or —C(O)C₁₋₄alkyl; and
R^{Xb}, at each occurrence, is independently hydrogen, C₁₋₄alkyl, C₁₋₂haloalkyl, or —C(O)C₁₋₄alkyl.

2. The method of claim 1, wherein the metaplastic cell or the dysplastic cell is contacted with a salt of the compound of formula (I).

3. The method of claim 2, wherein the salt of the compound of formula (I) is a salt of formula (I-A),

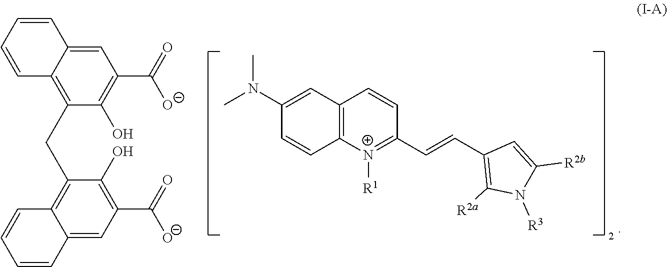

4. The method of claim 1, wherein R¹ is methyl.

5. The method of claim 1, wherein R^{2a} and R^{2b} are each methyl.

6. The method of claim 1, wherein R³ is unsubstituted phenyl.

7. The method of claim 1, wherein the compound of formula (I) is pyrvinium.

8. The method of claim 1, wherein the metaplastic cell or the dysplastic cell is located within a living organism.

9. The method of claim 1, wherein the pre-cancerous mucosa comprises a dysplastic cell.

10. (canceled)

11. The method of claim 9, wherein the dysplastic cell is positive for CD133 expression, CD166 expression, and/or Trop2 expression.

12. The method of claim 9, wherein the dysplastic cell is a dysplastic gastric cell, a dysplastic esophageal cell, a dysplastic pancreatic cell, a dysplastic colon cell, or a dysplastic ovarian cell.

13. (canceled)

14. The method of claim 1, wherein the pre-cancerous mucosa comprises a metaplastic cell.

15. The method of claim 14, wherein the metaplastic gastric cell is positive for CD133, CD166, and/or Trop2 expression.

16. The method of claim 14, wherein the metaplastic cell is a metaplastic gastric cell, a metaplastic esophageal cell, a metaplastic pancreatic cell, a metaplastic colon cell, or a metaplastic ovarian cell.

17. (canceled)

18. A method for reducing the risk of cancer development in a living organism containing a metaplastic cell or a dysplastic cell, the method comprising:
administering to the living organism a compound of formula (I), or a salt thereof: 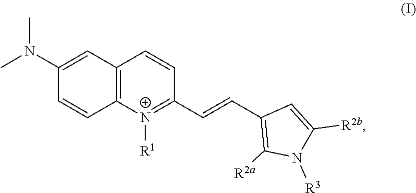
wherein:
R¹ is C₁₋₆alkyl;
R^{2a} and R^{2b} are each independently C₁₋₆alkyl; and
R³ is C₆₋₁₂aryl, wherein R³ is optionally substituted with 1-5 R^{X};
R^{X}, at each occurrence, is independently C₁₋₆alkyl, C₁₋₄haloalkyl, halogen, cyano, —N(R^{Xa})₂, —OR^{Xb}, —SR^{Xb}, —C(O)R^{Xb}, —CO₂R^{Xb}, —C(O)N(R^{Xa})₂, —SO₂R^{Xa}, -L²-Y², —O-L^{X}-Y^{X}, —S-L^{X}-Y^{X}, or —N(R^{Xa})-L^{X}-Y^{X};
L^{X}, at each occurrence, is independently C₁₋₆alkylene, C₂₋₆alkenylene, or C₂₋₆alkynylene;
Y^{X}, at each occurrence, is independently hydrogen, cyano, halogen, haloalkyl, —OH, —N(R^{Xa})₂, —OR^{Xb}, —SR^{Xb}, —C(O)R^{Xb}, —CO₂R^{Xb}, —C(O)N(R^{Xa})₂, or —SO₂R^{Xa};
R^{Xa}, at each occurrence, is independently hydrogen, C₁₋₄alkyl, or —C(O)C₁₋₄alkyl; and
R^{Xb}, at each occurrence, is independently hydrogen, C₁₋₄alkyl, C₁₋₂haloalkyl, or —C(O)C₁₋₄alkyl.

19. The method of claim 18, wherein a salt of the compound of formula (I) is administered to the living organism.

20. The method of claim 19, wherein the salt of the compound of formula (I) is a salt of formula (I-A),

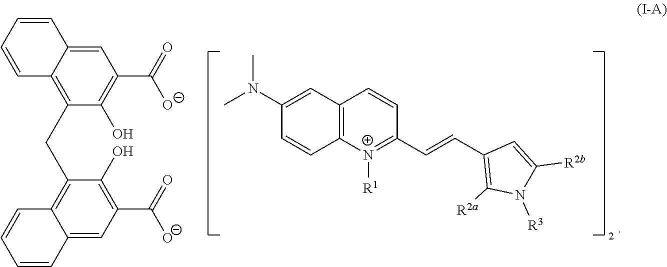

21. The method of claim 18, wherein the compound of formula (I) is pyrvinium.

22. (canceled)

23. The method of claim 18, wherein the living organism is a non-human animal.

24. The method of claim 18, wherein the living organism is a human.

25. (canceled)

26. The method of claim 18, the method further comprising administering a second agent to the living organism.

27-30. (canceled)
